(19) United States
(12) Patent Application Publication
(10) Pub. No.: US 2026/0016472 A1
(43) Pub. Date: Jan. 15, 2026

(54) DEVICES, SYSTEMS, AND METHODS FOR RAPID AND SCALABLE ELUTION OF ANALYTES FROM BEADS

(71) Applicant: ProQomics LLC, Pittsfield, MA (US)

(72) Inventor: Tamil Selvan Anthonymuthu, Stoneham, MA (US)

(21) Appl. No.: 19/261,464

(22) Filed: Jul. 7, 2025

Related U.S. Application Data
(60) Provisional application No. 63/669,200, filed on Jul. 9, 2024.

(51) Int. Cl. G01N 33/543; G01N 33/551; G01N 33/68

(52) U.S. Cl. CPC G01N 33/54386; G01N 33/551; G01N 33/6851

(57) ABSTRACT

Disclosed herein are devices, systems, and methods for efficiently eluting analytes (e.g., from one or more beads), allowing rapid and continuous analysis. Specifically disclosed is an apparatus with bead handling functions (e.g., bead handling devices such as bead picking units and/or vacuum pumps), one or more chambers and associated inlets, outlets, and/or fittings, pump units, devices (e.g., a rotating wheel) to move the one or more chambers to enable sample and/or analyte transfer and/or handling, one or more waste collection units, and one or more connections and/or connectors to analysis apparatuses and/or systems (e.g., mass spectrometers). A method disclosed herein employs repeatable cycles for: picking bead sets, relocating beads into chambers, inducing analyte migration into one or more elution solvents, and removing eluted beads to waste units. Each cycle can be repeated for various bead sets simultaneously, with solvent pumping and fluid flow aiding in analyte migration and bead removal.

### CROSS-REFERENCE TO RELATED APPLICATION

This application claims the benefit of U.S. Provisional Application No. 63/669,200, filed Jul. 9, 2024, which is hereby incorporated by reference in its entirety.

### FIELD OF THE DISCLOSURE

The present invention relates to devices, systems, apparatuses, and methods for biochemical analysis, high-throughput biological and/or biochemical assays, biomarkers, clinical diagnosis, chromatography, proteomics, multiplexed assays, and bead arrays. In particular, the present invention relates to devices, systems, apparatuses, and methods for the processing (including continuous and/or automated processing) and/or analysis of one or more eluted analytes. Such analytes may be eluted from one or more beads and/or types of beads, thereby enabling high-throughput qualitative and quantitative analysis of molecules, such as protein complexes, proteins, peptides, DNA, RNA, metabolites, lipids, carbohydrates, and synthetic small molecules such as drugs, drug molecules, and/or their precursors.

### BACKGROUND

Biomolecular analysis plays a pivotal role in various scientific disciplines, including molecular biology, clinical diagnostics, drug discovery, and precision medicine. Traditional methods for biomolecular analysis often face limitations in terms of sensitivity, specificity, reproducibility, and throughput. In recent years, bead-based assays have emerged as a powerful alternative, revolutionizing the way to detect, quantify, and analyze biomolecules. By leveraging the unique properties of microscopic beads as solid supports for biochemical reactions, bead-based assays offer unprecedented versatility and flexibility in experimental design. Due to the ease of development, quick handling time and specificity, these arrays have been successfully developed and deployed for several analytes. This includes oligonucleotides, proteins, polypeptides, post-translationally modified proteins, carbohydrates, lipids, antibodies, protein complexes, metabolites, and synthetic chemical molecules.

Generally, bead-based assays rely on the immobilization of specific ligands or probes onto microscopic beads, enabling the capture and detection of target biomolecules. The choice of ligands often depends on the nature of the assay and the analyte of interest, with common examples including antibodies, protein ligands, aptamers, oligonucleotides, and small molecules. Upon binding to the target molecules, the beads can serve as platforms for signal generation or readout, facilitating the detection and quantification of the analytes. Key steps in at least some bead-based assays include bead functionalization, sample incubation, washing to remove nonspecific binding, and signal detection. Detection methods can vary widely, ranging from fluorescence-based assays to electrochemical or mass spectrometry-based techniques.

The versatility of bead-based assays extends across various disciplines in biological research, encompassing applications such as immunoassays, protein-protein interaction studies, flow cytometry analysis, and nucleic acid detection. In immunoassays, bead-based formats, particularly in multiplex configurations, can enable simultaneous quantification of multiple proteins (e.g., cytokines, hormones) from a single sample, streamlining workflows in areas like immunology, allergy testing, and biomarker discovery. Furthermore, in protein-protein interaction studies, beads conjugated with specific proteins allow researchers to map interactions, unravel cellular signaling pathways, and identify potential drug targets. Integrating bead-based assays with flow cytometry facilitates cell surface marker analysis, providing valuable insights into cell phenotypes and immune cell populations. Additionally, beads are instrumental in nucleic acid detection, allowing the capture and detection of DNA or RNA molecules for applications such as genotyping, pathogen identification, and gene expression analysis.

Bead-based assays can therefore present a compelling alternative to conventional techniques like ELISAs (enzyme-linked immunosorbent assays) owing to several key advantages. Firstly, their multiplexing capability enables the simultaneous analysis of numerous analytes in a single experiment, translating to significant time and resource savings. Moreover, the efficient capture process and advanced detection methods employed in bead-based assays often result in improved sensitivity compared to traditional methods. Additionally, these assays typically require smaller sample volumes, making them ideal for precious biological samples or limited clinical specimens. The automation compatibility of bead-based assays can streamline workflows, enhancing throughput and minimizing manual intervention, thereby improving reproducibility. Furthermore, the modular nature of bead-based assays may allow for customization of target analytes and detection methods to suit specific research needs, adding to their flexibility and utility in diverse applications.

Affinity mass spectrometry, also known as affinity purification mass spectrometry, is an effective bead-based assay that can be used for isolating biological components from complex mixtures and analyzing them via mass spectrometry. This method typically involves enriching specific analytes or sets of analytes using microbeads, followed by elution and subsequent mass spectrometry analysis. Generally, affinity purification not only enriches the target analytes but also diminishes background noise during measurement, thereby enhancing the selectivity and specificity of the assay. In mass spectrometry, the mass-to-charge ratio (m/z) is used to determine the mass of the analyte, while the intensity reflects the relative amounts detected. Affinity mass spectrometry is often combined with liquid chromatography, where the eluted samples from beads are subjected to liquid chromatography step before mass spectrometry analysis. This step can help ensure the analytes are concentrated during mass spectrometry analysis.

The processing flow of affinity mass spectrometry often involves attaching antibodies against specific proteins or modified proteins to the microbeads, and incubation of affinity beads in the biological sample, where the formation of antibody-antigen complexes enriches the target analytes on the matrix. After incubation, the complex attached to the microbeads can be washed to eliminate non-specific interactions. The proteins enriched on the affinity matrix can then be eluted from the beads. Subsequently, the eluted sample may be loaded onto liquid chromatography mass spectrometry for further analysis.

In at least some instances, affinity mass spectrometry is a preferred method for highly reproducible and sensitive quantitative analysis in various research and analysis sectors. Automated instruments are now available to process the steps of affinity mass spectrometry, enabling quick and reproducible sample preparation. Typically, multiple samples are processed simultaneously in a multi-well format, such as a 96-well plate. Automated liquid/sample handling instruments can perform various steps, including sample transfer, addition of the microbeads, washing, and elution of the samples. In many cases, the eluted samples undergo further cleanup using techniques such as solid-phase extraction to remove components that may interfere with chromatography or mass spectrometry analysis performance. The eluates from solid-phase extraction are often concentrated using equipment, such as a speed vacuum or lyophilizer, before mass spectrometry analysis.

Affinity mass spectrometry can therefore offer several advantages for biomolecular analysis compared to orthogonal methods, such as ligand binding assays or techniques combining ligand binding with molecular biology methods like next-generation sequencing or polymerase chain reaction. These advantages can include a high dynamic range, excellent selectivity, and the ability to obtain structural information such as molecular weight and tandem mass spectrometry data for assessing chemical structure.

However, challenges exist with the aforementioned currently known bead-based assays, including the affinity mass spectrometry. For instance, the currently available devices, systems, apparatuses, and methods for bead handling, especially elution of the analytes from microbeads, are neither amenable for high throughput analysis nor directly connected to mass spectrometry.

In view of the foregoing, there is a significant need for devices, systems, apparatuses, and methods for processing and/or analysis of one or more eluted analytes, including analytes eluted from one or more beads. In particular, there is a need for continuous, automated, and/or high-throughput abilities to process and/or analyze such eluted analytes.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

In at least one embodiment of the invention, a bioanalytical instrument and an associated method is disclosed, which are capable of automatically processing beads pre-bound with analytes and displacing the analytes from the beads to send for further analysis. In at least one example, an apparatus is disclosed that can elute analytes from a plurality of multiplexed bead sets continuously. This can be achieved through placing multiple reusable chambers within the system and performing several parallel operations. In at least one embodiment, the number of chambers equals the number of parallel operations. The minimal set of operations may include packing the chamber with beads pre-bound with analytes, displacing the attached analytes from the beads into a moving elution buffer stream, and removal of eluted beads out of the chamber. For a given set of multiplexed beads, these operations are performed in sequence. Once the beads are removed from the chamber, the same chamber can be filled with the next set of beads and processed in a similar order. These steps enable the automated elution of analytes from a plurality of bead sets continuously.

In at least one embodiment, an apparatus enables the automated elution of analytes from a plurality of bead sets. This apparatus may comprise at least a bead handler that accepts the beads processed previously and transfers them to the bead processing unit of the apparatus, a bead feeder for transfer of the beads to bead processing, and the aforementioned bead processing, which may comprise one or more chambers to perform any of the operations described herein.

In at least a further embodiment, beads are transferred to the bead processing and/or one or more chambers without using a bead feeder. For instance, the beads could be transferred directly into, and placed within, one or more chambers.

In at least one embodiment, the apparatus further comprises one or more pumps and a waste collection unit, which is used to collect used beads and fluids. The bead processing also contains fluidic fittings that enable the chambers to connect to one or more pumps. In at least one embodiment, the apparatus further comprises one or more fluidic connections and/or connectors to transfer one or more eluted analytes to one or more analysis devices, such as, for instance, a mass spectrometer.

In at least one embodiment, the bead handler comprises bead storage and a bead manipulator. Previously processed beads can be stored in the bead storage in a particular order so that they can be selected according to the instructions for downstream processing. Bead storage may also comprise one or more areas, such as a bay, to place commonly used laboratory sample storage units, such as standard multi-well plates, standard vial holders, and the like. In at least one embodiment, multiple such areas and/or bays exist in order to store more than one such multi-well plates and/or vial holders. Non-limiting examples of standard multiple well storage units include: 48 well plate, 96 well plate, and 384 well plates. In at least one embodiment, areas or bays may be configured to store standard drum vials with different outer diameters and/or capacities.

In at least one embodiment, a bead manipulator is used to transport beads from one position and/or area of the apparatus and/or instrument to another. For instance, the bead manipulator can be a tool that uses magnets (e.g., magnetic-based bead manipulators) and/or suction (e.g., suction-based bead manipulators). In at least one example, magnetic-based bead manipulators comprise a paramagnetic element that is used to attract magnetic beads to the manipulator. The paramagnetic element can be released at the position where the bead needs to be discharged. In at least one example, suction-based manipulators comprise a tip into which the beads are sucked (e.g., into a wide opening micro tip). The beads are then released by releasing the suction pressure. In at least one embodiment, the bead manipulator can be a suction head fluidically connected to the inlet of one of the chambers enabling direct transfer of the beads to the chamber.

In at least one embodiment, a bead loader aids in the proper transfer of the beads from the bead manipulator to the bead processing unit.

In at least one embodiment, bead processing comprises a plurality of chambers, such as, for instance, at least three chambers, one or more of which may be identical. Such chambers may each have defined inlet and outlet openings, at least two fluidically attachable inlet fittings, at least three fluidically attachable outlet fittings, and at least one or more pumps to transfer essential liquids to and/or through the chambers and/or other portions of the apparatus and/or instrument.

In at least one embodiment, the chambers may be operably connected to fluid transport mechanisms, including but not limited to vacuum sources, pressure differentials, manually actuated syringes, and capillary-driven systems. In certain examples, the chambers may be fluidly coupled to a combination of pump-based and non-pump-based fluid transfer systems, enabling hybrid operation for flexible and controlled fluid handling.

The chambers in the aforementioned have at least one embodiment act as a reaction site wherein the beads pre-bound with the analytes are brought into contact with the analyte displacement reagent. The chambers also accommodate other bead-related processes, including filling with the beads selected and transferred from bead storage. The chambers are further configured to allow displacement of the beads from the chamber to one or more waste lines that fluidly connect to a waste collection or disposal unit. Thus, in at least one example, the chambers are configured to allow for the inflow and outflow of larger beads at one end and to allow the fluid flow at the other end. The inlet bore can therefore be larger in size to allow at least one bead at a time, while the outlet bore can be smaller to retain the beads inside the chamber. The chamber also has provisions that enable proper placement of fittings, such as connection fittings. Non-limiting examples of such fittings include, for instance, tapered seating for ferrule attachment, coupling, frits, custom fittings, leak free fittings, easily replaceable fittings and flanges to properly seat and engage the fittings.

In at least one embodiment, one or more chambers are placed on a closed system such as a wheel or a similar rotating mechanism. The wheel may be rotated using a mechanical drive such as a shaft attached to the center of the wheel or a gear system present in the wheel as well as in, for instance, a rotating shaft. Such rotating wheels can move and/or adjust the one or more chambers into different positions such that different bead processing and/or analysis steps can be performed (e.g., filling of beads in the chamber, eluting of analytes from the beads in the chamber, removing of eluted beads from the chamber, washing and/or cleaning up of the chamber, preparation of the chamber for a next set of beads, etc.). Alternative positioning portions and/or aspects may be used, such as, for instance, a moving conveyor belt, a fixed shaft, other rotational aspects and/or devices, stationary belts, servo motors, micro-stepping motors, gears, and the like. Further, movable fittings may be used in at least one embodiment with fixed and/or stationary shafts, belts, and the like.

In certain embodiments, one or more chambers are positioned in a fixed or stationary configuration, while one or more fluidic fittings are configured to rotate or move relative to the chambers to facilitate specific fluidic operations. For instance, during a chamber filling operation, fluidic fittings connected to a bead feeder and a vacuum pump may be selectively coupled to the chamber. In another example, for an elution process, a fluidic fitting connected to an elution reagent source may be engaged with the chamber. The fluidic fittings may be actuated and engaged either independently—each having a dedicated actuation and engagement mechanism—or collectively, wherein multiple fittings are mounted on a shared movable structure, such as a rotating wheel, conveyor mechanism, gear assembly, or other translation or indexing systems.

In at least one embodiment, the apparatus also includes at least two inlet fittings that allow the flow of fluids through the pumps and at least three outlet fittings that also allow the transfer of the fluid through the chamber. These fittings can be engaged before the start of each bead processing step and disengaged before further processing and/or analysis (e.g., during the movement of the chambers attached to the rotary wheels). The engaging and disengaging of the fittings can be performed using one or more fasteners or fluid connectors known in the art, such as, for instance, mechanical or electrical-based fasteners, active and/or passive positioning mechanisms.

In at least one embodiment, the outlet fitting of one of the chambers is connected to an external analytical instrument such as a mass spectrometer. Other outlet fittings, including, in at least one example, at least two outlet fittings, may connect to a waste chamber and/or waste collection unit (e.g., through a vacuum pump).

In at least one embodiment, the apparatus and/or device includes at least one or more pumps or fluid transport channel to control the flow of the fluids to and out of one or more chambers. One of the pumps or the fluid transport channel may be used to mobilize the elution buffer from a storage system and/or area to one or more chambers. Another pump or the fluid transport channel can be used to pump washing buffer to one or more chambers. Various types of pumps can be used, such as, for example, isocratic, binary, quaternary, and the like. Another pump that can be used is a vacuum pump that removes waste fluid from one or more chambers (e.g., after elution of one or more analytes from the beads). The fluid transport channel may include one or more connections configured to interface with non-pump-based fluid transport systems or with separate lines coupled to a pump. In some embodiments, the channel may support selective integration of both pump-based and non-pump-based inputs to enable flexible fluid handling.

In at least one embodiment, the one or more chambers are identical. In at least a further embodiment, the various inlet fittings on the one or more chambers are identical. In at least an additional embodiment, the various outlet fittings are identical. In various embodiments, only one pair of fittings can be engaged to the chamber's inlet and outlet. The outlet of the chamber connected to the bead feeder may, for instance, always be connected to the fittings that are connected to the waste collection unit (e.g., through the vacuum pump). Similarly, the chamber connected to one or more wash pumps may also always be connected to the waste collection unit (e.g., through the vacuum pump). Further, a chamber connected to the pump pumping the elution solvent may always be connected to the external analytical apparatus (e.g., a mass spectrometer).

In at least one embodiment, a waste collection unit is used to store the waste generated from various steps of the processing and/or analysis described herein (e.g., used elution solvent).

In at least one embodiment, further aspects and/or portions of the apparatus and/or device include mechanical and/or electrical systems that perform highly precise movement of various parts, including any part described herein, control systems of such parts, and computing devices and/or software that regulates the control system, an electronic/digital user interface, and storage systems (e.g., bead storage). Implementations of the techniques described may include hardware, a method or process, or a computer tangible medium.

In at least one embodiment, a software is used to control the instrument. The software stack comprises of a user interface, to receive operation commands from the user, a data handling middleware for displaying and processing information related to the current state of the process, prompts for the user and related information as well as a low-level firmware control for hardware motion control and sensing. In at least one embodiment, the instrument uses standard communication and data transfer protocols required to interface with any upstream instrument as well as any downstream analytical instrument such as a mass spectrometer.

In at least one embodiment, a method is disclosed herein for continuous, automated elution of bound analytes from a plurality of multiplexed bead sets and/or a plurality of beads. In at least one example, the method utilizes the apparatus and/or device described herein. The method may comprise successive steps, such as, for instance, repositioning of the beads from a specific storage location to an empty chamber or packing of the chamber with beads, displacing the analytes to the elution buffer or elution, removal of the eluted beads from the chamber or unpacking, and/or washing of the chamber after removal of eluted beads. In at least one embodiment, the method is repeated for every set of multiplexed beads stored in the bead storage. Further, in at least one embodiment, the method can be performed in parallel for different bead sets.

In at least one embodiment, the aforementioned packing of the chamber with beads is performed using a bead manipulator and a bead feeder. The bead manipulator can pick the beads from the bead storage and position them into the bead feeder. The beads may then be transferred to one of the chambers connected to the bead feeder. The transfer may happen due to gravity or with the help of one or more pumps (e.g., suction pumps). After transferring all the beads from a single set to the chamber connected to the bead feeder, the feeder may be disengaged, removed from the aforementioned connection to the chamber, and the chamber may be moved away from the feeder (e.g., via rotation using a wheel on which the chamber is attached). For instance, in embodiments in which a rotating wheel is used, the wheel may rotate one-third of a turn to move the chamber away from the bead feeder. This turn may position the filled chamber to a second position on the wheel and, simultaneously, position another empty chamber to the first position; that is, the position nearest to the bead feeder. Now in the second position, the inlet of the chamber can be engaged with the fittings attached to the pump transporting the elution buffer, and the outlet can be engaged with the outlet connected to the external analytical instrument (e.g., mass spectrometer). Upon connecting, the pump can transport the elution solvent enabling the contact between the beads and elution solvent. This will displace one or more analytes from the beads into the flowing elution solvent flow. While elution is occurring, in at least one embodiment, a different set of multiplexed beads can be packed into a new chamber located at the first position via, for instance, the bead feeder. After this, one or more fittings, connections, and/or connectors can be disengaged and then, the wheel can rotate (e.g., another one-third turn) to bring the first chamber into a third position, bring the second chamber into the second position, and bring the third chamber into the first position. At this third position, the eluted beads can be removed with the help of washing solvent pumped through a washing pump, and the washed mixture may be directed to the waste collection unit. While the beads in the first chamber are removed at the aforementioned second position on the wheel, the beads in the second chamber will be eluted, and the first chamber will be filled with a new bead set at the first position (e.g., via the bead feeder). These steps can be repeated to complete the analysis of all the samples present in the bead storage or programmed for analysis.

In at least one embodiment, the analytes displaced from the beads may comprise one or more biological compounds, including, for instance, one or more nucleic acids, one or more proteins, one or more protein complexes, one or more modified proteins, one or more peptides, one or more carbohydrates, one or more metabolites, one or more lipids, one or more enzymes, one or more protein fragments, one or more nucleic acid fragments, one or more carbohydrate fragments, one or more lipid fragments, one or more pharmacological substances, one or more drugs, one or more pro-drugs, one or more small molecules, one or more chemical compounds, one or more biological compounds, one or more atoms, one or more molecules, one or more viral particles, one or more cells, and combinations thereof. In at least one embodiment, each bead within a given bead set may be associated with one or more compounds (including, for instance, chemically distinct molecules), such as, for example, one or more nucleic acids, one or more proteins, one or more protein complexes, one or more modified proteins, one or more peptides, one or more carbohydrates, one or more metabolites, one or more lipids, one or more enzymes, one or more protein fragments, one or more nucleic acid fragments, one or more carbohydrate fragments, one or more lipid fragments, one or more pharmacological substances, one or more drugs, one or more pro-drugs, one or more small molecules, one or more chemical compounds, one or more biological compounds, one or more atoms, one or more molecules, one or more viral particles, one or more cells, and combinations thereof. In at least one embodiment, one or more analytes displaced from the beads are analyzed by one or more analysis devices (e.g., a mass spectrometer). In at least one embodiment, the method may include analyzing the eluted analytes for quantification and/or identification purposes. For instance, each bead may include at least one chemically distinct molecule that is used for bead identification. In at least one embodiment, the eluted analytes are further processed and/or analyzed for downstream applications, including, but not limited to, sequencing, proteomics, metabolomics, lipidomics, and combinations thereof.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an apparatus for the elution, processing, and/or analysis of analytes from beads, according to at least one embodiment of the present disclosure.

FIGS. 2A-2D show a bead feeder, according to at least one embodiment of the present disclosure.

FIG. 3 shows the structure of one or more chambers that may hold beads, according to at least one embodiment of the present disclosure.

FIGS. 4A-4J show different possible structures of one or more chambers, according to at least one embodiment of the present disclosure.

FIGS. 5A-5D show different possible fasteners and/or fastening arrangements for one or more chambers, according to at least one embodiment of the present disclosure.

FIGS. 6A-6L show different possible arrangements of chambers, including both moveable and immovable chambers, according to at least one embodiment of the present disclosure.

FIGS. 7A-7F show various inlet fittings and/or fitting arrangements on the one or more chambers, according to at least one embodiment of the present disclosure.

FIGS. 8A-8F show various outlet fittings and/or fitting arrangements on the one or more chambers, according to at least one embodiment of the present disclosure.

FIGS. 9A-9B show further types of inlet fittings and/or fitting arrangements on the one or more chambers, according to at least one embodiment of the present disclosure.

FIGS. 10A-10B show further types of outlet fittings and/or fitting arrangements on the one or more chambers, according to at least one embodiment of the present disclosure.

FIGS. 11A-11F show various possible mechanisms for engaging inlet and/or outlet fittings to the one or more chambers, according to at least one embodiment of the present disclosure.

FIGS. 12A-12B show further types of connections and/or connectors for the one or more chambers, according to at least one embodiment of the present disclosure.

FIG. 13 shows one step in a method for the elution, processing, and/or analysis of analytes from beads, according to at least one embodiment of the present disclosure.

FIG. 14 shows a further step in a method for the elution, processing, and/or analysis of analytes from beads, according to at least one embodiment of the present disclosure.

FIG. 15 shows a still further step in a method for the elution, processing, and/or analysis of analytes from beads, according to at least one embodiment of the present disclosure.

FIG. 16 shows a still further step in a method for the elution, processing, and/or analysis of analytes from beads, according to at least one embodiment of the present disclosure.

FIGS. 17A-17F show various possible arrangements of beads inside one or more chambers, according to at least one embodiment of the present disclosure.

FIGS. 18A-18D show various possible steps in a method for the elution, processing, and/or analysis of analytes from beads with respect to different chambers and/or chamber positions, according to at least one embodiment of the present disclosure.

FIG. 19 shows a system for elution, processing, and/or analysis of analytes from beads, according to at least one embodiment of the present disclosure.

FIG. 20 shows a sample experiment in which four beads, each conjugated with a specific antibody, were reacted with a biological sample, according to at least one embodiment of the present disclosure.

FIGS. 21A-21C show mass spectra obtained with various different bead arrangements inside one or more chambers, according to at least one embodiment of the present disclosure.

Reference to various embodiments does not limit the scope of the invention. Figures represented herein are not limitations to the various embodiments according to the invention and are presented as exemplary illustration of the invention.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment,” “an embodiment,” “an example embodiment,” etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may affect such feature, structure, or characteristic in connection with other embodiments whether or not explicitly described.

In several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a,” “an,” and “the” are intended to include the plural forms as well, unless the context clearly indicates otherwise. It should also be noted that, in some alternative implementations, the functions and/or acts noted may occur out of order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise,” “comprises,” and “comprising” are to be interpreted inclusively rather than exclusively. Likewise, the terms “include,” “including,” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of.” Although having distinct meanings, the terms “comprising,” “having,” “containing,” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

### Definitions

The following is a non-exhaustive and non-limiting list of terms used herein and their respective definitions.

The term “analyte,” at least as used herein, refers to a chemical entity that may be detected and/or analyzed by one or more analytical methods. Non-limiting examples of “analytes” include, for instance, one or more atoms and/or portions of atoms, one or more molecules and/or portions of molecules, complexes of molecules, and the like. Thus, various biological compounds, including, but not limited to, nucleic acids, carbohydrates, proteins, and lipids, may all be “analytes,” In at least some instances, a plurality of similar analytes and/or types of analytes can be considered to be the same “analyte” or category of “analyte.”

The term “bead,” at least as used herein, refers to a particle used in experimental and/or laboratory investigations. “Beads” may be a variety of different sizes. Further, “beads” may be a variety of shapes, including, but not limited to, spherical, approximately spherical, oval, approximately oval, ovoid, and the like. Non-spherical shapes are also possible, such as, for instance, tubular shapes (e.g., nanotubes), rod-like shapes (e.g., nanorods), etc. Many “beads” are microparticles and/or nanoparticles. Specific “beads” may have one or more surfaces that are reactive and can be reacted with several compounds (e.g., one or more molecules) to derive specific characteristics.

The term “compound” refers to a substance formed from one or more chemical elements, arranged together in any proportion or structural arrangement. The one or more chemical elements may be either naturally occurring and/or non-naturally occurring. As used herein, the term “biological compound” refers to a compound of biological origin and/or having one or more effects on a subject's local and/or systemic biological functions. Accordingly, “compounds” or “biological compounds” include, as non-limiting examples, various proteins (e.g., growth factors, hormones, enzymes), nucleic acids, and pharmaceutical products (e.g., drugs, prodrugs). The term “drug” generally refers to a medicine or other substance that has a physiological effect when introduced into a subject. The term “prodrug” generally refers to a biologically and/or chemically inactive compound that can be metabolized by a subject to produce a drug.

The term “high throughput,” at least as used herein, refers to the ability of a device, system, or method to handle a large volume of tasks or samples in a short amount of time. Thus, “high throughput” devices, systems, and methods are those that quickly and/or efficiently process a plurality of tasks and/or a plurality of samples.

The term “microparticle,” as used herein, generally refers to a particle having a diameter, such as an average diameter, from about 1 micron to about 1,000 microns, preferably from about 50 microns to about 500 microns, more preferably from about 100 microns to about 200 microns. The microparticles can have any shape. Microparticles having a spherical shape are generally referred to as “microspheres.”

The term “molecular weight” generally refers to the relative average molecular weight of a bulk polymer or protein, unless otherwise specified. In practice, molecular weights can be estimated or characterized using various methods including, for example, gel permeation chromatography (GPC) or capillary viscometry. GPC molecular weights are reported as the weight-average molecular weight (MW), as opposed to the number-average molecular weight (MN). Capillary viscometry provides estimates of molecular weight as the inherent viscosity determined from a dilute polymer solution using a particular set of concentration, temperature, and solvent conditions.

The term “multi-well plate,” which may also be referred to as “microplate,” “microtiter plate,” “microwell plate,” or simply “multi-well,” refers to a piece of experimental and/or laboratory equipment, which may be disposable or reusable, that contains multiple wells for the storage and/or deposition of one or more fluids under experimental investigation. Thus, “multi-well plates” can be used in a variety of common biological, chemical, and/or biochemical experimental protocols and/or assays, including, but not limited to, enzyme-linked immunosorbent assays (ELISAs). Non-limiting examples of the number of wells in a “multi-well plate” include 6, 12, 24, 48, 96, 384, and 1536. The wells in a “multi-well plate” can hold varying amounts of liquid, such as, for instance, anywhere from nanoliters to milliliters of liquid. Further, a skilled artisan will recognize that “multi-well plates” may be used for powders, solids, and the like. Different shapes of “vials” exist, including, but not limited to, cylindrical, tubular, rectangular, conical, and the like.

The term “nanoparticle,” as used herein, generally refers to a particle having a diameter, such as an average diameter, from about 10 nanometers (nm) up to but not including about 1 micron, preferably from 100 nm to about 1 micron. The particles can have any shape. Nanoparticles having a spherical shape are generally referred to as “nanospheres.”

The term “particle size,” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

The term “peptide” refers to a polymer of amino acid residues. The amino acid residues may be naturally occurring and/or non-naturally occurring. The terms “polypeptide,” “peptide,” and “protein” are used interchangeably herein. The terms apply to, for instance, amino acid polymers of one or more amino acid residues, an artificial chemical mimetic of a corresponding naturally occurring amino acid, naturally occurring amino acid polymers, and non-naturally occurring amino acid polymers.

The term “vial,” which may also be referred to as a “test tube,” refers to a vessel or bottle for the storage and/or deposition of one or more fluids under experimental investigation. “Vials” can store specific dosages of liquids, including one or more dosages (e.g., single-dosage vials, multi-dosage vials, and the like). Further, a skilled artisan will recognize that “vials” may be used for powders, solids, and the like. Different shapes of “vials” exist, including, but not limited to, cylindrical, tubular, rectangular, and the like.

Further, unless otherwise noted, technical terms are generally used according to conventional usage. Aspects of the disclosed methods employ, unless indicated specifically to the contrary, conventional methods of chemistry, biochemistry, organic chemistry, molecular biology, microbiology, recombinant DNA techniques, genetics, immunology, and/or cell biology, many of which are described below solely for the purpose of illustration. Such techniques are explained fully in technical literature sources. General definitions of common terms in the aforementioned fields, including, for instance, molecular biology, may be found in references such as, e.g., Krebs et al., Lewin's Genes X, Jones & Bartlett Learning (2009) (ISBN 0763766321); Rédei, Encyclopedic Dictionary of Genetics, Genomics, Proteomics and Informatics (3rd ed.), Springer (2008) (ISBN: 1402067532); Ausubel et al., Current Protocols in Molecular Biology, John Wiley and Sons (updated July 2008) (ISBN: 047150338X); Ausubel et al., Short Protocols in Molecular Biology: A Compendium of Methods from Current Protocols in Molecular Biology (2nd ed.), Wiley-Interscience (1989) (ISBN 0471514705); Glover, et al., DNA Cloning: A Practical Approach, Vol. I-II, Oxford University Press (1985) (ISBN 0199634777); Anand et al., Techniques for the Analysis of Complex Genomes, Academic Press (1992) (ISBN 0120576201); Hames et al., Transcription and Translation: A Practical Approach, Oxford University Press (1984) (ISBN 0904147525); Perbal et al., A Practical Guide to Molecular Cloning (2nd ed.), Wiley-Interscience (1988) (ISBN 0471850713); Kendrew et al., Encyclopedia of Molecular Biology, Wiley-Blackwall (1994) (ISBN 0632021829); Meyers et al., Molecular Biology and Biotechnology: A Comprehensive Desk Reference, Wiley-VCH (1996) (ISBN 047118571X); Harlow et al., Antibodies: A Laboratory Manual, Cold Spring Harbor Laboratory Press (1988) (ISBN 0879693746); Coligan et al., Current Protocols in Immunology, Current Protocols (2002) (ISBN 0471522767); Annual Review of Immunology; articles and/or monographs in scientific journals (e.g., Advances in Immunology); and other similar references.

### Elution and Analysis of Analytes

The described innovation details an apparatus for high throughput analysis of various analytes attached to the surface of the beads.

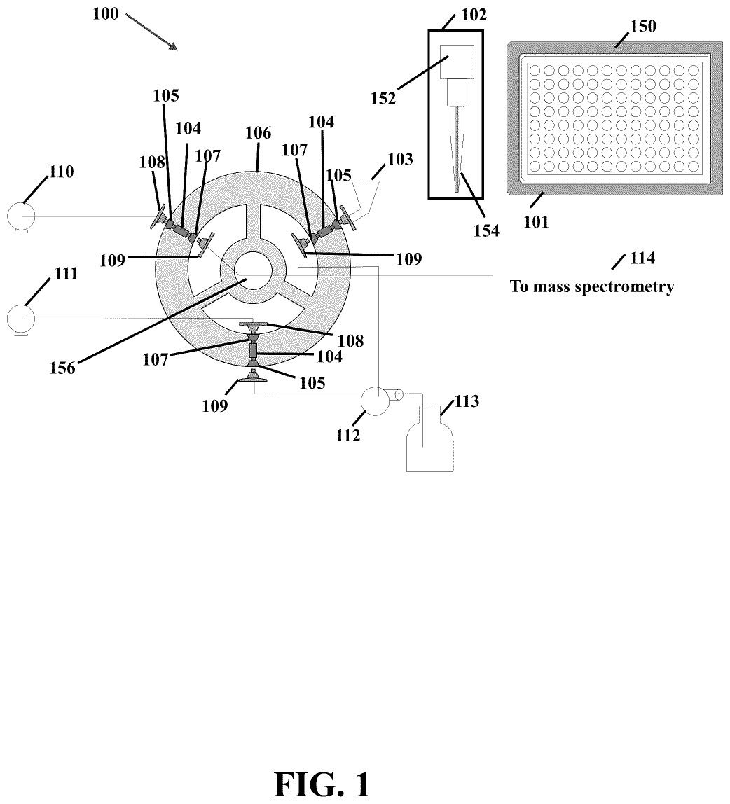

Turning now to FIG. 1, a schematic representation of an analytical apparatus 100 according to at least one embodiment of the disclosure is shown. Bead storage 101 stores one or more beads, which may be disposed in any form of storage, including, but not limited to, vials, plates (e.g., multi-well plates), and the like. As a non-limiting example, a multi-well plate 150 is shown disposed in bead storage 101. Although multi-well plate 150 is shown as a 96-well plate, it should be appreciated that any multi-well plate, with any number(s) of wells, can be disposed in bead storage 101. For instance, multi-well plate 150 may contain one well, 4 wells, 8 wells, 10 wells, 12 wells, 20 wells, 24 wells, 30 wells, 48 wells, 96 wells, any number of wells from 1-100, any number of wells from 1-200, any number of wells from 1-300, any number of wells from 1-400, any number of wells from 1-500, or any number of wells more than 500. Thus, as a non-limiting example, a multi-well plate 150 may be a 48-well plate, a 96-well plate, a 384-well plate, or the like. Multi-well plates may be constructed of any suitable material, including, for instance, glass, any type of plastic, etc. Wells in one or more multi-well plates may be in a range of sizes and hold a range of volumes, including, for instance, a minimum of 0.1 μL and a maximum of 100 μL, 200 μL, 300 μL, 0.5 mL, 1 mL, 1.5 mL, 2 mL, 4 mL, 5 mL, 10 mL, or more than 10 mL. Further, in at least one example, more than one multi-well plate may be disposed in bead storage 101 in any arrangement, such as, for instance, arranged horizontally, arranged vertically, and/or stacked, and/or combinations thereof. The usage of more than one multi-well plate may further enhance the throughput of apparatus 100.

Additionally, multi-well plate 150 is not restricted to any specific type of plate. For example, plate 150 may be one or more of: a polymerase chain reaction (PCR) plate, a quantitative PCR (qPCR) plate, an elution plate, a micro-elution plate, a micro-plate, an imaging plate (e.g., for luminometers and/or fluorometers), an immunoassay plate, a titration plate, a micro-titration plate, a sonication plate, an ultra-sonication plate, a deep-well plate, a cell culture plate, etc. The plate 150 may be one or more of: thick-walled, thin-walled, skirted, semi-skirted, sub-skirted, non-skirted, having a rigid frame, having a flexible frame, etc. Further, plate 150 may contain any one or more shapes of individual wells, such as, for instance, circular, cylindrical, square, rectangular, and/or any type of polygon. One or more wells in the plate 150 may also be recessed below the surface of the plate 150 and/or the bead storage 101.

As stated above, bead storage 101 may, in at least one embodiment, contain one or more vials and/or vial storage devices and/or systems instead of, or in addition to, one or more multi-well plates (e.g., multi-well plate 150). Such vials may be, for instance, any type of vial holding beads, solutions, and/or samples, including, for example, drum vials. One or more vials may be in any one or more shapes, such as, for instance, circular, cylindrical, square, rectangular, and/or any type of polygon. Vials may be constructed of any suitable material, including, for instance, glass, any type of plastic, etc. One or more vials may be in a range of sizes and hold a range of volumes, including, for instance, a minimum of 0.1 μL and a maximum of 100 μL, 200 μL, 300 μL, 0.5 mL, 1 mL, 1.5 mL, 2 mL, 4 mL, 5 mL, 10 mL, or more than 10 mL. Usage of more than one vial and/or vial storage device and/or system may further enhance the throughput of apparatus 100.

In at least one embodiment, the plate 150 (and/or one or more vials and/or vial storage devices and/or systems) may be seated and/or nested in the bead storage 101 via, for instance, one or more raised surfaces, one or more depressed surfaces, one or more bays, or the like.

In at least one embodiment, bead storage 101 may further comprise one or more temperature controls and/or temperature-controlled portions and/or units. Such temperature controls and/or temperature-controlled portions and/or units may be disposed within the bead storage 101 such that they regulate the temperature and/or humidity of any beads, solutions, and/or samples (e.g., any analytes) disposed within bead storage 101. As described above herein, the aforementioned beads, solutions, and/or samples (e.g., any analytes) may be disposed within, for instance, multi-well plate 150 (and/or vials and/or vial storage devices and/or systems), which itself is disposed within bead storage 101.

Temperature controls and/or temperature-controlled portions and/or units may regulate temperature by increasing the temperature, decreasing the temperature, and/or keeping the temperature the same according to one or more settings and/or user preferences (e.g., keeping the temperature at room temperature). Accordingly, in at least one example, an air-conditioned unit is used to reduce the temperature within bead storage 101. Such temperature reduction may be achieved by any suitable method, including, for instance, a chilled block or cold block on which the multi-well plate 150 rests. Such a chilled block or cold block may have one or more indentations so that one or more wells of multi-well plate 150 (and/or one or more vials) fit and/or are nested within these indentations. Temperature and/or humidity regulation may be achieved by any suitable method, including, in the non-limiting example of an air-conditioned unit, using one or more air compressors, using one or more batteries, and/or using one or more electricity-powered devices and/or systems.

In at least one embodiment, bead manipulator 102 comprises one or more arms 152 for picking up one or more beads, which, as described above herein, can be disposed within bead storage 101. The one or more beads can be, for instance, magnetic beads. Thus, bead manipulator 102 may further comprise a movable rod and a removable tip 154. In at least one example, bead manipulator 102 facilitates the manipulation of magnetic beads equipped with a magnetic core. Alternatively or additionally, in some embodiments, the bead manipulator 102 could be a pipetting unit with any opening tip and/or pipetting tip (e.g., a wide opening tip). In some embodiments, the bead manipulator 105 could be a needle connected to a pump or fluid/solid transfer unit such as pumps or suction units. In at least one embodiment, bead manipulator 102 is capable of movement in one or more of three dimensions (e.g., one or more of the x, y, and z directions) to facilitate the retrieval of beads from bead storage 101, including, for instance, one or more specific wells and/or one or more specific vials therein, and repositioning of such beads in the appropriate downstream units, devices, and/or systems, as described further below herein.

In at least one embodiment, bead manipulator 102 moves and/or repositions one or more beads into bead feeder 103, which is therefore downstream of the bead manipulator 102. Bead feeder 103 enables the transfer of beads from the bead storage 101 to subsequent bead processing devices, systems, and/or steps, as described further below herein. In an alternative embodiment the bead feeder 103 could be an Bead feeder 103 can be fluidically connected to a chamber 104. The chamber 104 may comprise one or more separate portions and may be shaped in any suitable shape for the movement of one or more beads through the chamber 104 and/or the one or more portions thereof. Further, the chamber 104 comprises an opening 105 for the connection of the bead feeder and the transfer of beads from the bead feeder 103. In at least one example, the opening 105 has a broader cross-section than one or more portions of the chamber 104 to enable ease of transfer of the beads. Further, the opening 105 may be connected to a fitting 108.

In at least one embodiment, apparatus 100 comprises one or more such chambers 104. For instance, and as shown, three chambers 104 are used. Each such chamber may be attached to a wheel or a rotating unit 106. The wheel 106 may be any shape that accommodates the one or more chambers 104 and can rotate, including, but not limited to, a circular shape, or move. A skilled artisan will appreciate other shapes are possible, including any type of polygonal shape. Positionally, the one or more chambers 104 are disposed around the wheel 106 such that one end of each of the chambers are directed and/or aligned towards a center 156 of the wheel. As a non-limiting example, the one or more chambers 104 can be positioned such that they are orthogonally arranged to a circumference of the wheel 106.

In at least one embodiment, the circular wheel 106 can be rotated about its axis (which may be disposed at, for instance, center 156) to alter the positioning of the chambers 104. Each chamber 104 may comprise an opening 107 that is disposed along a length of the chamber and at an opposite end of the chamber as opening 105. The opening 107 may be connected to a separate fitting 109. In at least one example, one or more openings 105 and one or more openings 107 are identical. In at least another example, one or more fittings 108 are identical to one or more fittings 109. Thus, in at least some examples, one or more openings 105 are interchangeable with one or more openings 107, and further, one or more fittings 108 are interchangeable with one or more fittings 109.

In at least one embodiment, apparatus 100 further comprises one or more pumps. As a non-limiting example, at least three pumps 110, 111, and 112 are used. Each such pump may be of the same type, or of a different type, than any of the other pumps. For instance, pumps 110 and 111 may be capillary flow pumps, while pump 112 may be a vacuum pump. One or more such pumps 110, 111, and 112 may be connected to one or more chambers 104, and/or one or more portions thereof, such as, for instance, fittings 108 and/or fittings 109.

In the non-limiting example where pumps 110 and 111 are capillary pumps, both such pumps can be connected to the inlet of one or more chambers 104 via the fittings 108. Such connection may facilitate fluid transfer into the chambers 104. Further, pump 112, which, as described above herein, may be a vacuum pump, can be connected to one or more fittings 109 to draw out liquid (e.g., outlet liquid) from the chambers 104, and through the fittings 109 to a waste chamber 113.

In at least one example, the waste chamber 113 may be fluidly connected to the pump 112 and one or more of the chambers 104, and/or one or more portions thereof (e.g., fitting 109).

In at least one embodiment, at least one pump 110 is used to pump an elution solvent, and at least one pump 111 is employed to transfer wash liquids to one or more of the chambers 104. As shown, pump 110 can pump an elution solvent through fitting 108, which can flow through opening 105. Pump 111 can also pump one or more wash liquids through fitting 108. Any suitable solvent used for elution can be used, including, but not limited to, organic solvents, polar solvents, solvent mixtures, alcohols, water, acids, ion paring agents, acetonitrile, and the like. Further, any suitable wash liquid used for washing one or more beads, including any type of beads (e.g., magnetic beads) can be used, including, but not limited to, one or more buffers, water, and the like.

In at least one embodiment, and as shown, one chamber 104 is connected to bead feeder 103. Pump 111, which can be used for pumping one or more wash liquids, is linked and/or fluidly connected to the vacuum pump 112, which can be used to extract liquid to waste chamber 113. Further, and as shown, another chamber 104 is connected to both pump 110 (e.g., via fitting 108), which can be used to pump an elution solvent, and to an input to a mass spectrometry device and/or system 114 (e.g., via fitting 109).

### Bead Feeder

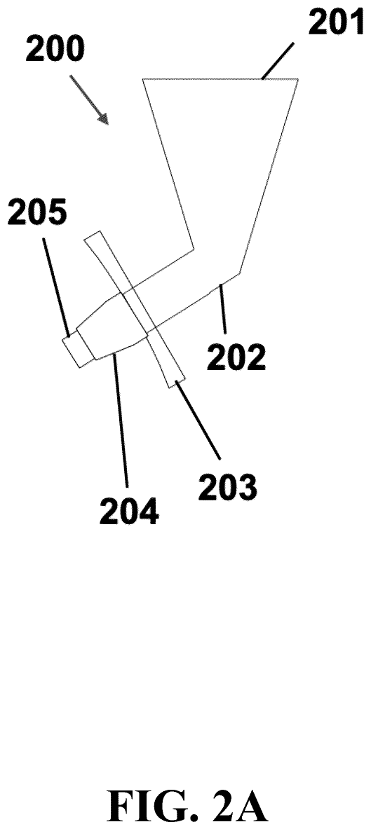

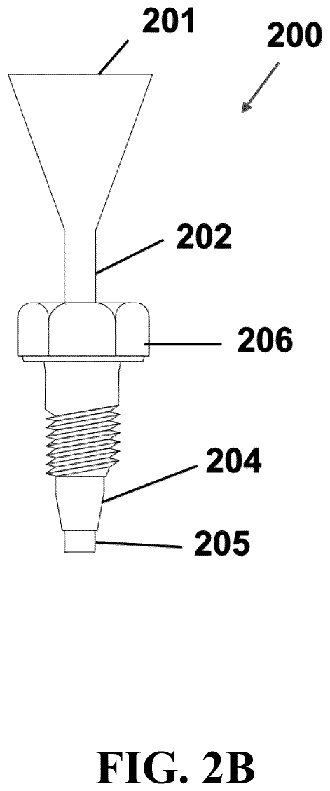

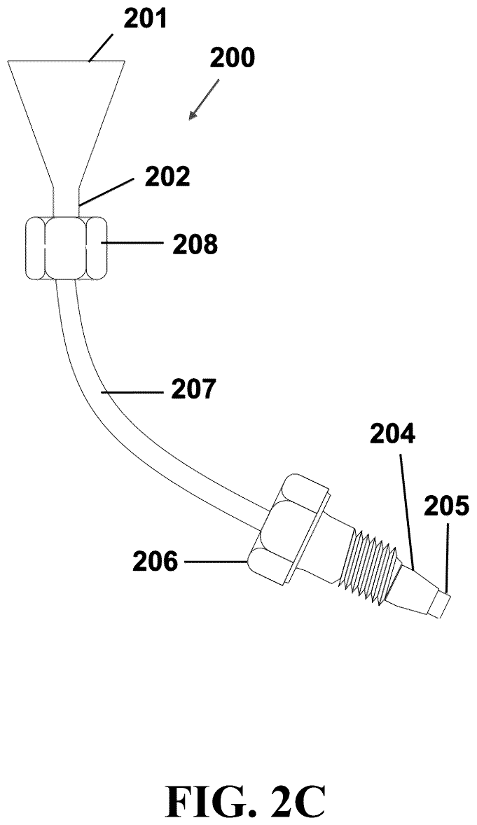

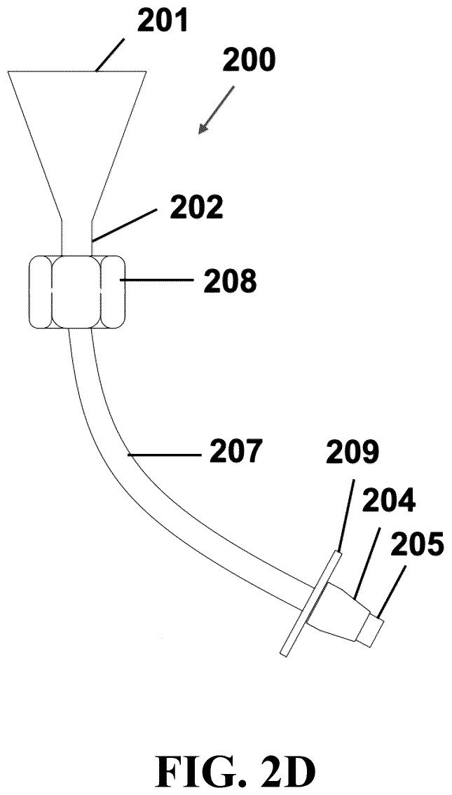

Turning now to FIGS. 2A-2D, a bead feeder 200 according to at least one embodiment is disclosed. It should be appreciated that bead feeder 200 may be similar to, or the same as, any bead feeder described herein, including, but not limited to, bead feeder 103. Specifically, FIG. 2A is a schematic diagram of the bead feeder 200, which is roughly in the shape of a funnel. One or more beads, as described above herein, can be transferred and/or moved from bead storage 101 and deposited into opening 201, which is physically and/or fluidly connected to stem 202. The stem 202, in turn, is physically and/or fluidly connected to a fluidic fitting, which, in at least one embodiment, comprises a ferrule 204, and a tube or pilot 205. In at least one embodiment, the fluidic fittings of the bead feeder are supported by flange 203, which is physically and/or fluidly connected to both the tube or pilot 205, and one or more fittings as described above herein (e.g., fittings 108 and/or 109). As depicted in FIG. 2A, the stem 202 of the feeder 200 may be angled. Any specific angle may be used in order to enable flow of the beads through the bead feeder 200. Additionally or alternatively, and as shown in FIG. 2B, the stem 202 of the feeder 200 may be straight or substantially straight. The angle of the stem, in at least one embodiment, is determined by the positioning of the one or more chambers 104. Several embodiments of bead feeder 200, which may be the same and/or different, may be used to enable easy connection with the one or more chambers 104.

Further, as shown in FIG. 2B, bead feeder 200 comprises connector 206 instead of flange 203. The connector 206 may be physically and/or fluidly connected to stem 202 with any type of connection mechanism, including, but not limited to, screws, tabs, pins, mating mechanisms, and the like. The bead feeder shown in FIG. 2B also depicts a tube or pilot 205, as well as a ferrule 204. In at least one embodiment, connector 206 may be fixed such that it is not freely moveable along stem 202.

In at least one embodiment, and as shown in FIG. 2C, bead feeder 200 comprises of a connector 206 which can be movable using a tube 207 and a coupling 208. For instance, the connector 206 can be moved to any point or area along the tube 207 and attached via the coupling 208. The coupling 208 may employ any type of connection mechanism, including, but not limited to, screws, tabs, pins, mating mechanisms, and the like.

In at least one embodiment, and as shown in FIG. 2D, bead feeder 200 also comprises a tube 207 and a coupling 208. However, fitting 209 is shown instead of connector 206. The fitting 209 may be, for instance, a flange or a flange-type fitting. In some embodiments the flange may contain one or more screws id easy engagement or disengagement.

In the aforementioned embodiments, the size of the bead feeder 200 and any tubing thereof (e.g., tube 207) can be chosen and/or determined (e.g., by a user) by considering one or more factors, including, but not limited to, the size of the beads used, the number of beads used, the type of beads used, and the like. Further, the size, number, and/or type of beads used may be different for different types of analysis desired or required by a user. Additionally, the size of mouth or opening 101 can be different depending on the size, number, and/or type of beads used. As a non-limiting example, opening 101 may be similar to, or the same as, or larger than, the common well size used in one or more multi-well plates, e.g., a 96-well plate. Further, the inner diameter of one or more portions of the bead feeder 200, including, for instance, stem 202, may be 10% or more larger than the size of the individual beads and up to 90% larger to facilitate easy and quick transfer of beads. In at least one embodiments, the stem 202 may be large enough to transfer 2 or more beads together, for instance, the stem may 220% larger than the bead diameter to facilitate transfer of 2 beads together, 320% larger than the bead diameter to transfer 3 beads, 550% larger than the bead diameter larger than bead diameter to transfer 5 beads at the same time, and the like.

### Chamber Structure

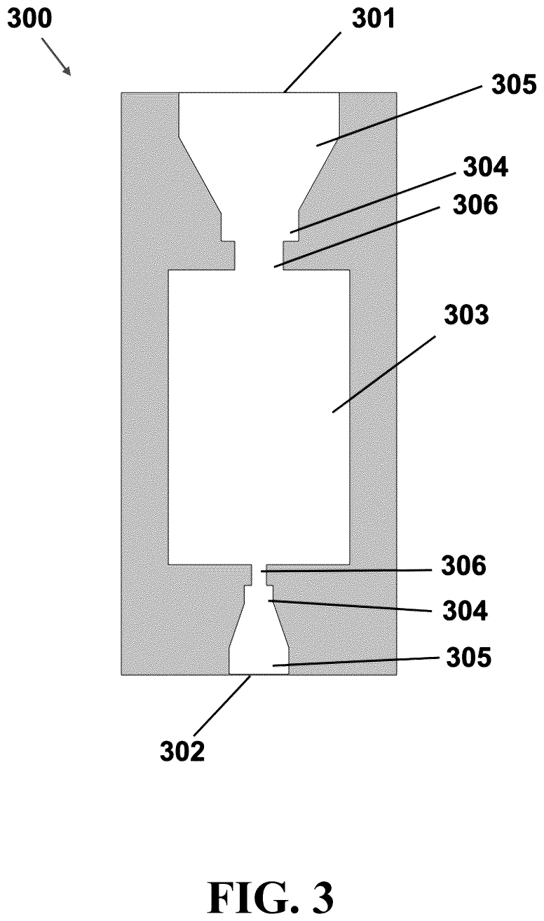

FIG. 3 shows the structure of one or more chambers 300, which may be similar to, or the same as, any other chambers described herein (e.g., chambers 104). The chamber 300 comprises inlet opening 301 and outlet opening 302. In at least one embodiment, the chamber further comprises a cylindrical hollow structure 303 disposed between the inlet 301 and the outlet 302. The hollow structure 303 is connected to a pilot 304 and a tapered ferrule seat 305 through a pore 306 on both ends. In other words, the pilot 304, the ferrule seat 305, and the pore 306 fluidly connect the hollow structure 303 to both the inlet 301 and the outlet 302. In this at least one embodiment, the pore size of the inlet opening 301 is larger than the outlet opening 302 to allow the inflow of larger beads, whereas on the outlet opening 302, the pore size is adjusted to retain the beads inside the chamber or one or more portions thereof (e.g., the hollow structure 303). The dimensions of the chamber can vary depending upon the size, number, and/or type of beads used. In some cases, the size of the beads may be determined by the volume of elution buffer required for complete discharge of one or more analytes from the beads. A skilled artisan will recognize that several elution methods require varying volume and/or contact time for complete displacement of the analyte. For example, elution based on pH changes may require very small volumes, while in cases of competition-based elution, the elution can be a function of competitive substrate at the binding site and the time of exposure. In at least one example, the eluted samples are subjected to direct mass spectrometry analysis (e.g., via mass spectrometry 114. Thus, in this at least one example, the maximum amount of competitive substances may be limited by the mass spectrometer tolerance.

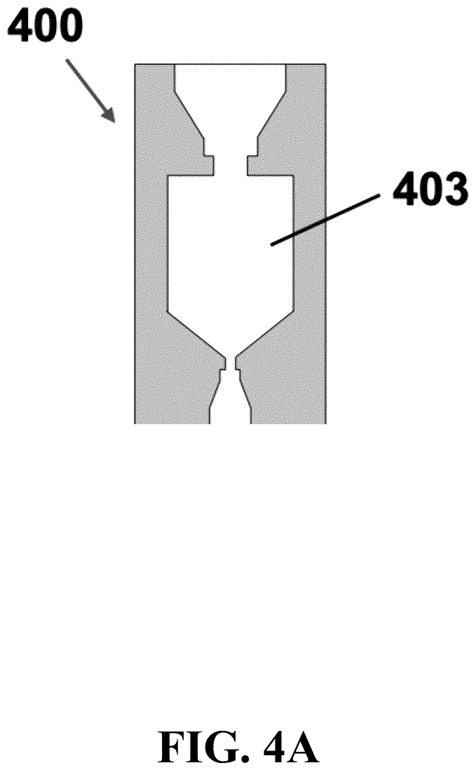

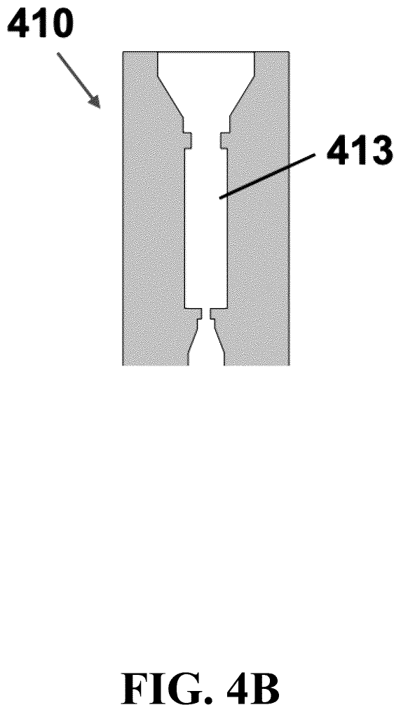

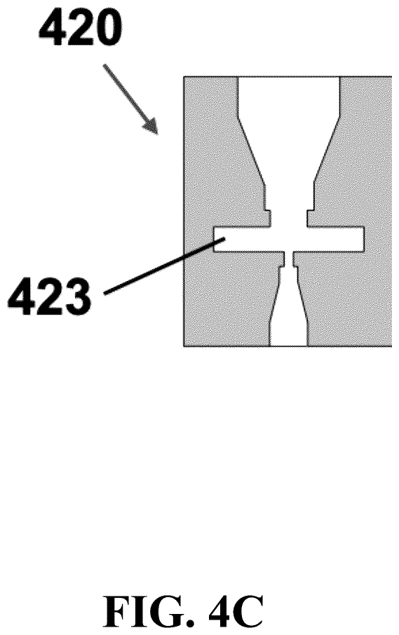

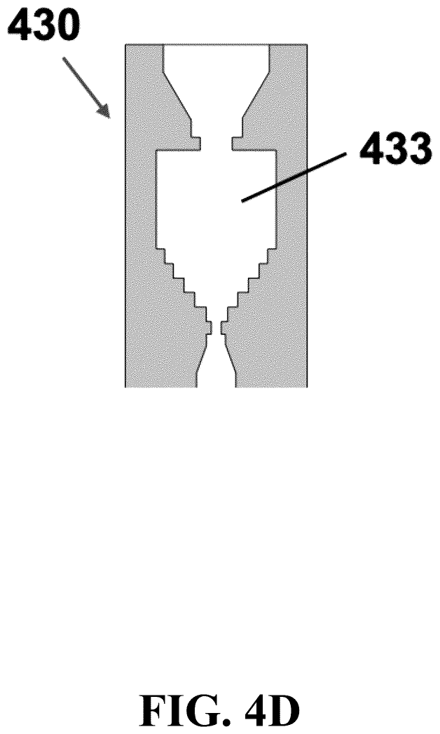

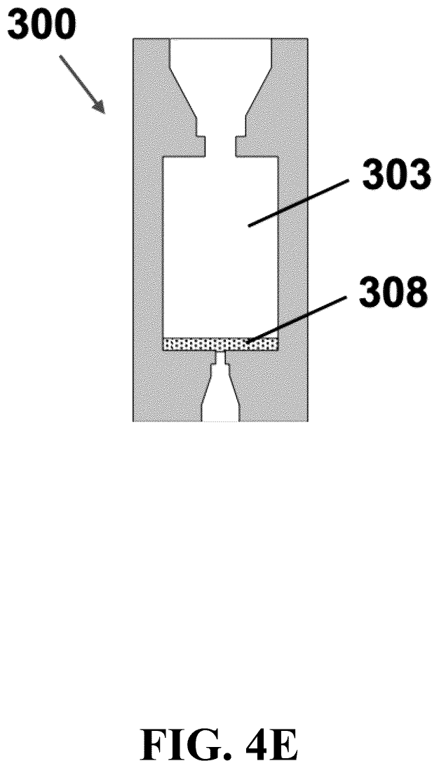

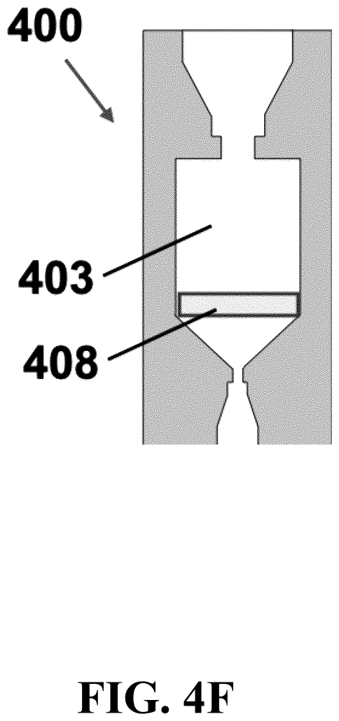

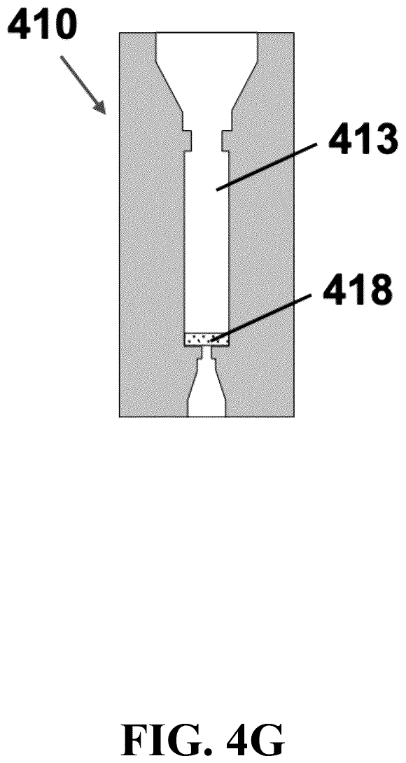

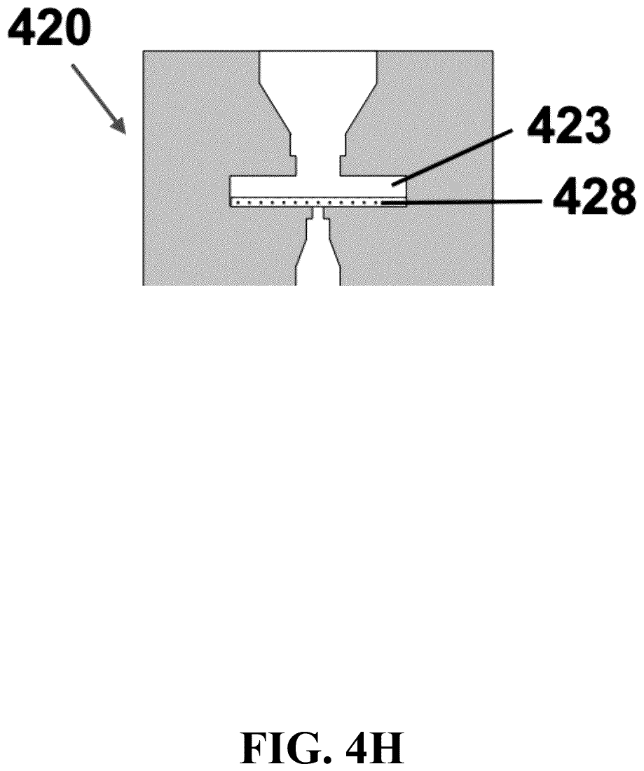

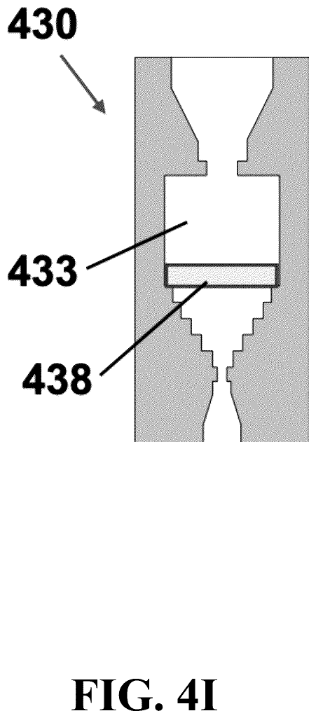

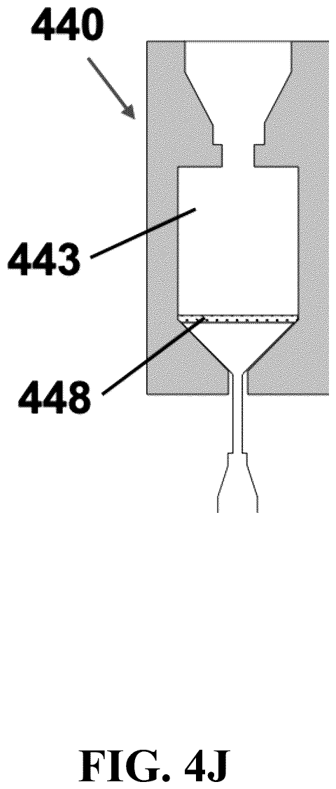

While a cylindrical chamber may be optimal for one or more reactions relating to elution from the beads, the chamber may be designed in different forms. For instance, FIGS. 4A-4H depict several geometrical designs of the chambers, in at least one embodiment. FIG. 4A depicts a chamber 400 with a hollow structure 403 in the shape of cylinder at the top portion and then a conical structure at the bottom portion, tapering until the diameter of the hollow structure 403 is substantially equal to the bore size. In another embodiment, as depicted in FIG. 4B, chamber 410 has a hollow structure 413 that is substantially rectangular in size, for instance, such as the length of the hollow structure can be much larger than the diameter of the structure. For example, the height of the hollow structure can be more than 5 times the diameter. In at least one embodiment shown in FIG. 4B, such a geometry could enable better separation of analytes from one or more beads. In another embodiment, the diameter of the chamber and/or one or more portions thereof, such as the hollow structure, can be much larger, for example, more than 5 times. As depicted in FIG. 4C, the geometry of the chamber 420 is such that hollow structure 423 is substantially rectangular along an axis perpendicular to the direction of flow of the beads. Such a structure can be used to form a monolayer or bilayer of the beads (e.g., when the beads collect in the structure 423). In another embodiment, shown in FIG. 4D, chamber 430 comprises a hollow structure 433 that has a bottom portion (that is, a portion fluidly connected to the outlet) that is made up of several cylinders of the same axis and different diameters, of increasingly narrow diameters. Thus, as shown, the diameter of the top of the structure 433 will be larger than the bottom portion and the cylinders forming such bottom portion, as presented in FIG. 4D.

The embodiments described in FIG. 3 and FIGS. 4A-4D can rely on the size of the outlet opening to retain the beads inside. In other words, the diameter of the outlet opening is at least 10% smaller than the diameter of the beads. A skilled artisan will appreciate that beads are often not of equal size and/or shape. In such cases, the flow of the fluids out of the chamber (e.g., any of the chambers described herein, including those shown in FIGS. 3 and 4A-4D) can be disrupted due to the positioning of one or a set of beads close to the outlet opening section. Thus, in at least some embodiments, the addition of a fixed frit, that is, a sieve structure smaller than the size of the beads but large enough to allow free flow of liquid, can alleviate this issue. FIGS. 4E-4I show the chambers depicted in FIG. 3 and FIGS. 4A-4D, except with a baffle located at the bottom of the chamber. Specifically, FIG. 4E shows chamber 300 and hollow structure 303, with the addition of baffle 308. Similarly, FIG. 4F shows chamber 400 and hollow structure 403, with the addition of baffle 408. Additionally, FIG. 4G shows chamber 410 and hollow structure 413, with the addition of baffle 418. Further, FIG. 4H shows chamber 420 and hollow structure 423, with the addition of baffle 428. Still further, FIG. 4I shows chamber 430 and hollow structure 433, with the addition of baffle 438. In all of the above embodiments showing the various chamber shapes and/or arrangements, the volume of the chamber is fixed to a predetermined volume. Thus, the shape and/or structure of the chamber may be changed based on the experimental conditions (e.g., to accommodate one or more shapes, sizes, and/or types of beads, one or more types of elution buffers, one or more types of analytes, etc.). In such cases, the frit or baffle (e.g., any of the baffles described herein) can be made to move inside the chamber and/or hollow structure. As a non-limiting example, FIG. 4J shows chamber 440 and hollow structure 443 with moveable frit or baffle 448.

In at least one embodiment, the volume, and/or dimensions of the chamber (or chambers, if multiple chambers are used) can depend on the experimental conditions, such as the size of the beads and/or the number of beads. For instance, the volume of the chamber required for the number of beads can be calculated based on the formula V_{c}=NπD_{b}³/3.84. Here, Ve is the volume of the chamber, N is the number of beads, and D_{b} is the diameter of the beads. This equation is calculated using the formulas, the volume of the bead with a diameter D_{b} is equal to πD_{b}³/6, and the volume of N number of beads will be NπD_{b}³/6. The effective volume can be decreased due to the unfilled spaces between the packed beads. The beads can be filled in different arrangements, such as cubic, hexagonal close-packed, or random close-packed. In at least one example, the proportion of the void volume or the empty volume of each packing may be estimated at 47.6%, 26%, and 36%, respectively. This leads to a packing efficiency of 0.524, 0.74, and 0.64, respectively. In bead packing conditions described herein, random close packing of the beads is also possible. Therefore, in at least one embodiment, the effective volume of N number of beads would be equivalent to (NπD_{b}³/6)/0.64 or NπD_{b}³/3.84. As per the equation, the minimal volume of the chamber required for 100 numbers of 100-micron beads would be 0.082 mm³, while this volume will be increased to 0.645 mm³ for 100 numbers of 200-micron beads and 5.24 mm³ for the same number of 400-micron beads. The diameter and the height of the chamber can be calculated based on the formula V_{c}=πD_{c}²H_{c}. D_{c} is the diameter of the chamber, and He is the height of the chamber.

Alternatively, the maximum number of beads that can be used in a fixed chamber of known diameter (D_{c}) and known height (H_{c}) can be calculated using the following equation N=0.96D_{c}²H/D_{b}³. As per this calculation, a chamber of 1 mm×1 mm (Diameter×Height) can accommodate 960 numbers of 100-micron beads, whereas a 2 mm×2 mm chamber can accommodate about 15360 beads. While the numbers will be reduced to 1920 and 240 beads for beads of size 200 microns and 400 microns.

Chamber Connections, Connectors, and/or Fittings

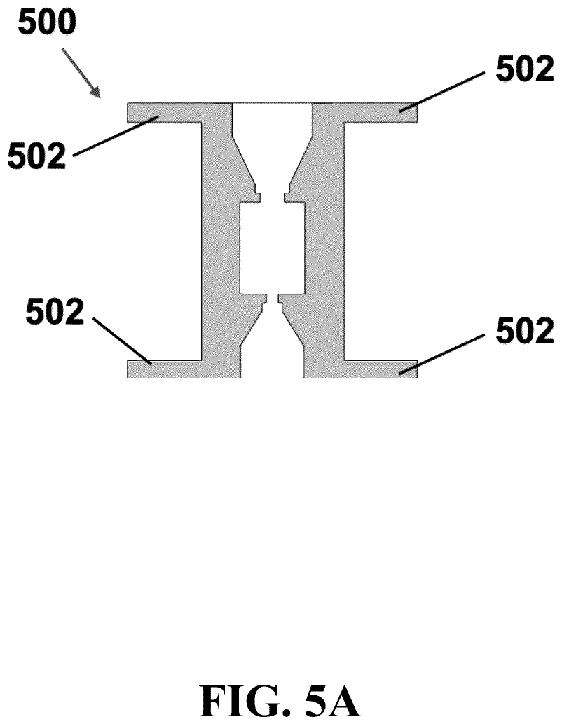

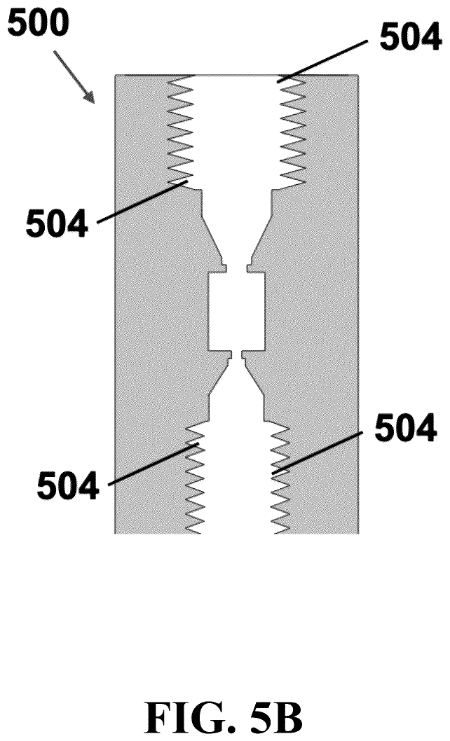

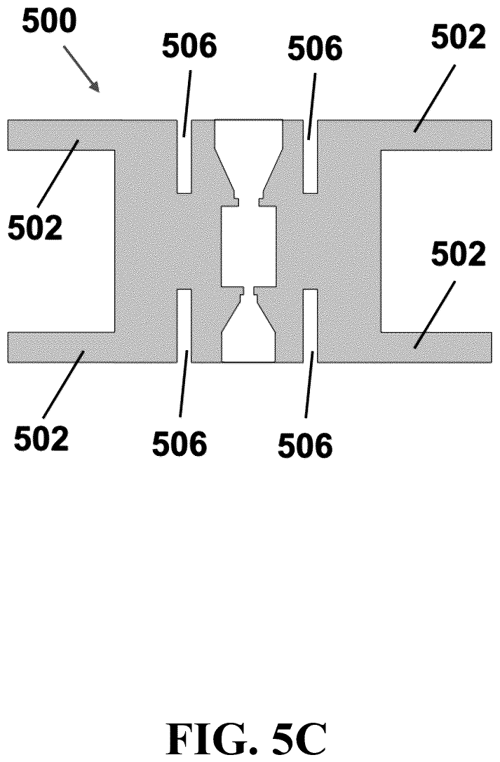

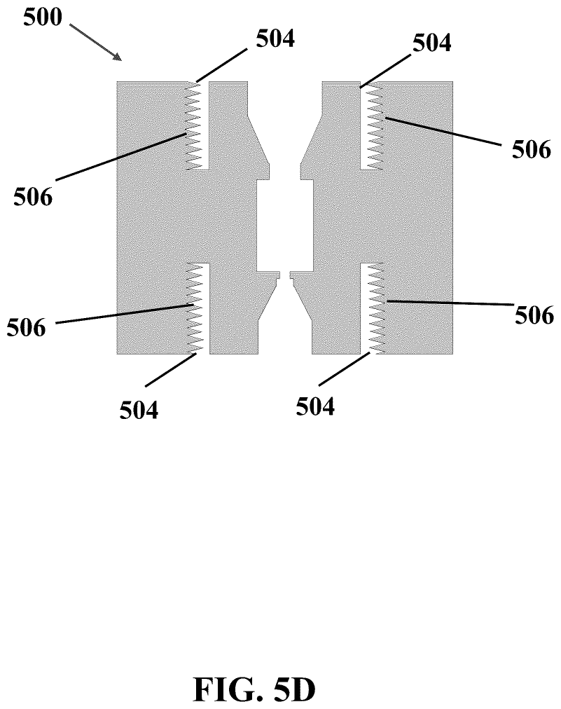

In certain embodiments described herein, the one or more chambers (e.g., any chamber described herein) need to be automatically attached and detached to one or more respective fittings to complete their functions. This may be enhanced by introducing supporting elements or aspects in the chamber setup. One non-limiting example could be including a flange on both ends of the chambers. As depicted in FIG. 5A, chamber 500 comprises one or more flanges 502 that can provide additional support for the continuous engaging and/or disengaging of the chamber, as well as ease of operation. In at least another embodiment, a secured fitting may be obtained by screwed type joints. Such joints can provide several advantages in terms of ease of operation, accessibility, and flexibility. In some embodiments the flange may include a threaded grove for a screw or nut to enable easy engagement and disengagement of fluidic fittings. FIG. 5B shows a chamber 500 shows a female-type threaded joints 504. As shown in FIG. 5C, along with the flanges 502, the chamber 500 may also have some additional alignment features/grooves 506, of which only a subset are labeled for convenience, to guide the fittings to the proper position and reduce the potential damages to the fitting during the automated engaging and disengaging process. Thus, in the aforementioned at least one embodiment, one or more flanges and/or one or more alignment features/grooves may be located around the fitting parts.

In another embodiment, as depicted in FIG. 5D, the one or more threads 506 can be included in the one or more alignment features/grooves 506 to release the load from the fluidic fittings. It should be appreciated that the described embodiments of the fittings are not intended to be all-inclusive. Other standard fittings used for automation can also be implemented in one or more chambers. Some non-limiting examples may include groove and tongue fittings, quick snap fittings, and any other fittings providing removable connections.

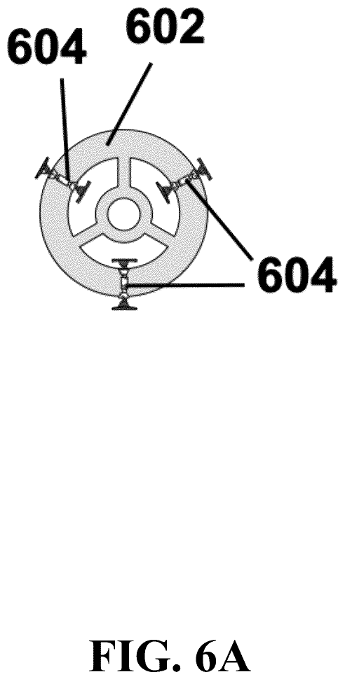

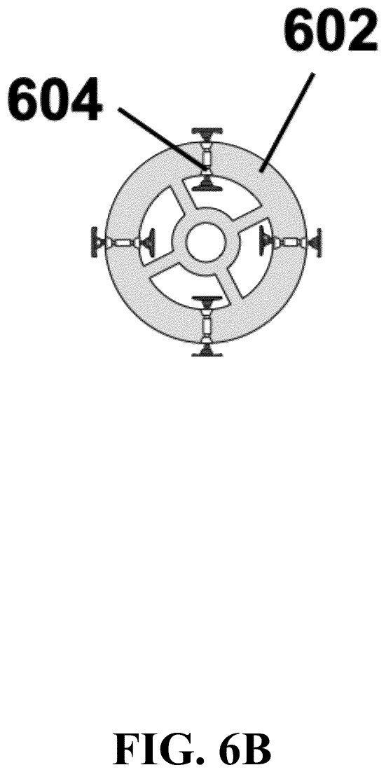

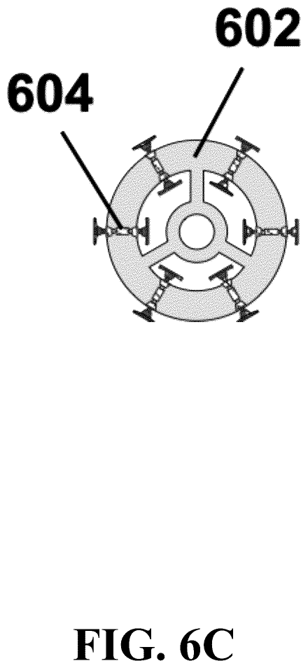

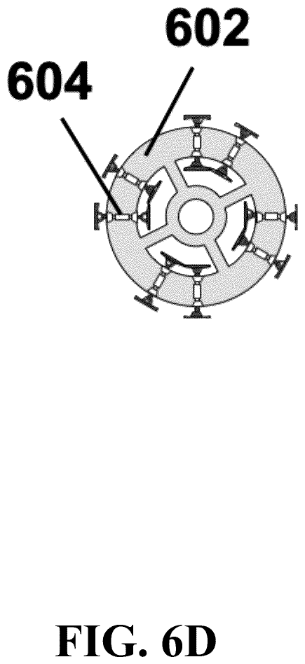

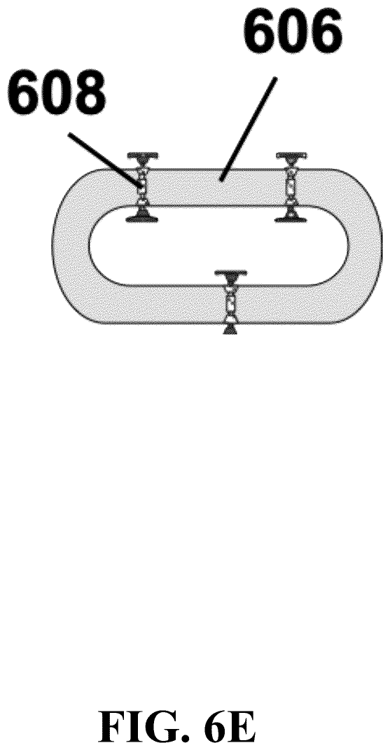

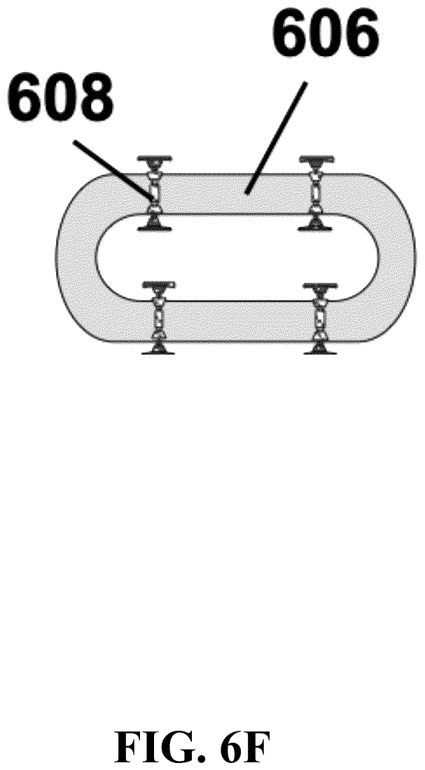

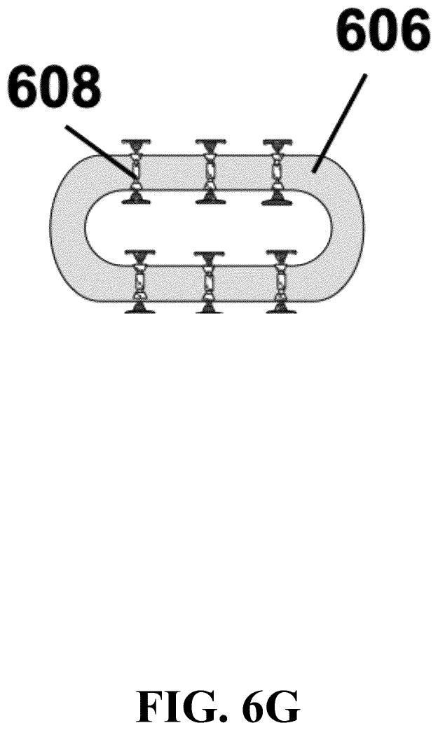

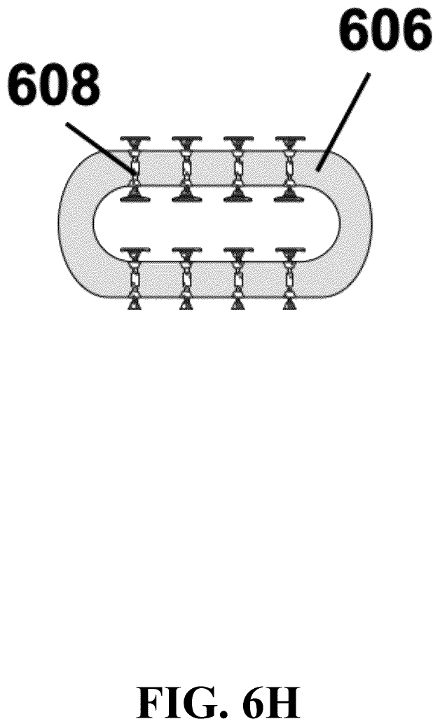

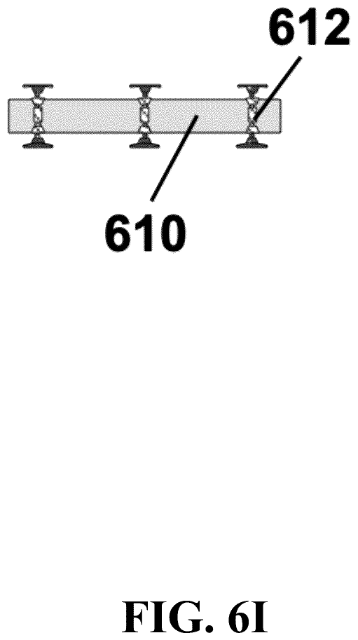

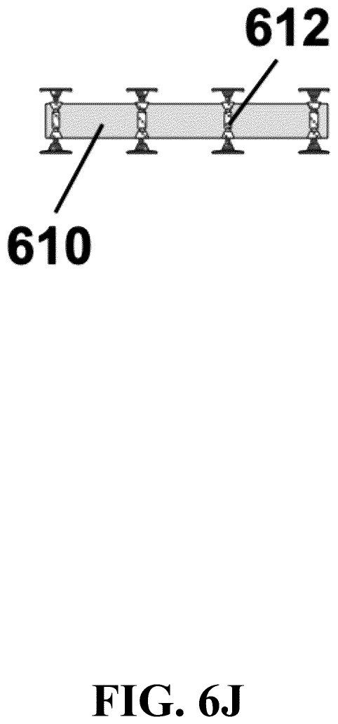

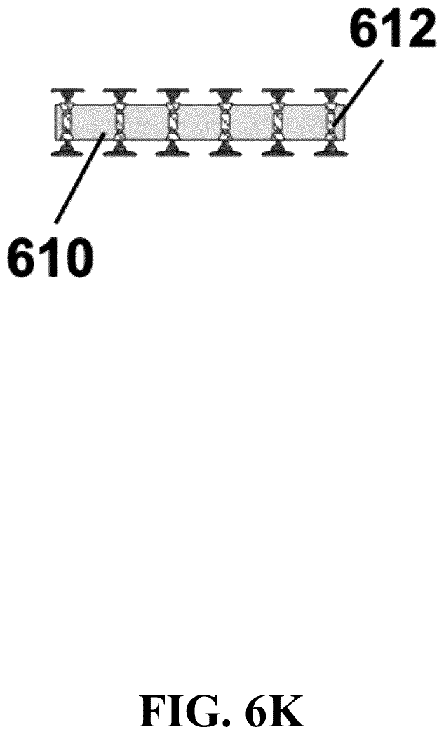

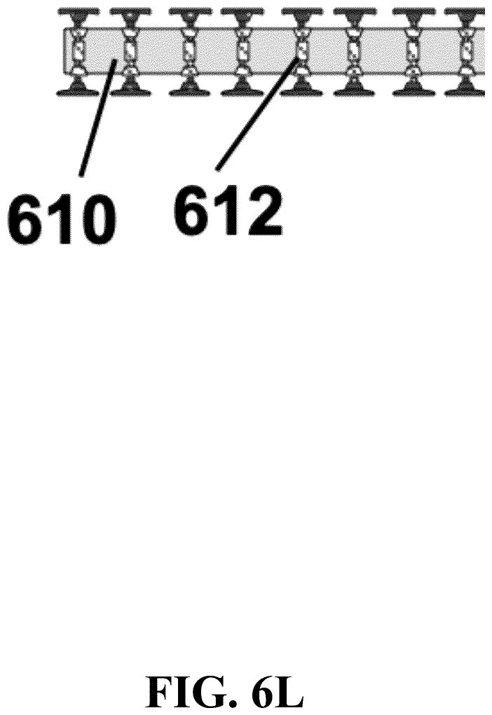

In at least one embodiment, the minimum number of chambers (which may, in at least one example, be identical) needed for the performance of the apparatus (e.g., any apparatus described herein, such as, for instance, apparatus 100) is 3. However, the number of chambers can also be increased to provide additional processing steps. This warrants multiple chambers attached to the rotating wheel or moving belt or in the fixed fixture (e.g., as shown in FIG. 1). For instance, FIG. 6A depicts rotating wheel 602, which may be similar to, or the same as, any such wheel described herein (e.g., wheel 106), with three chambers 604. One or more such chambers 604 may be similar to, or the same as, any one or more such chambers described herein. In at least another embodiment, the wheel 602 can have 4 chambers 604, as depicted in FIG. 6B. It should be appreciated that only one such chamber 604 is labeled for the sake of convenience. In at least one example, these 4 chambers 604 are identical, which can provide various advantages, including, for instance, offering better positioning of chambers for some of the operations. For example, the bead feeding position can be kept straight upward, and the bead removal position can be kept straight downward facing. This may help gravity-based operations in these positions. In another example the chamber may be positioned parallel to the rotating axis, such that all chambers maintain the same upright orientation throughout the rotation.

In certain embodiments, 2 chambers can be used for performing the same process step, which may increase the speed of operation. For instance, FIG. 6C depicts a wheel 602 with 6 chambers 604, only one of which is labeled for convenience. In at least one example, any two chambers placed adjacent to each other can be used to perform the same or similar function. Similarly, FIG. 6D depicts a wheel 602 with 8 chambers 604, only one of which is labeled for convenience. These 8 chambers can provide twice the operational space of a wheel having 4 chambers.

While the identical chambers described in several embodiments may be placed in rotatable wheels in order to provide ease of operation (e.g., through an attached motor of a gear system), several other arrangements of the chamber and/or wheel may be employed. Turning now to FIGS. 6E-6H, various arrangements are shown in which chambers are placed on a rotating structure, for instance, an rounded rectangle or capsule or carousel-like structure. Specifically, FIG. 6E shows oval-shaped structure 606 with 3 chambers 608 disposed on the structure. Only one such chamber is labeled for the sake of convenience, but it should be appreciated that one or more such chambers 608 may be similar to, or the same as, any one or more other chambers described herein. Further, FIG. 6F shows structure 606 with 4 chambers 608, only one of which is labeled for the sake of convenience. Still further, FIGS. 6G-6H show structure 606 having 6 chambers 608 and 8 chambers 608, respectively. Advantages of the rounded rectangle shaped placement include, for instance, some or most of the operations being performed with the chambers placed in an identical orientation that is upright and/or a vertical up-down positioning. This can enable simple handling of the chambers, one or more fittings, and/or ease of transfer of the beads and/or elution of analytes therefrom.

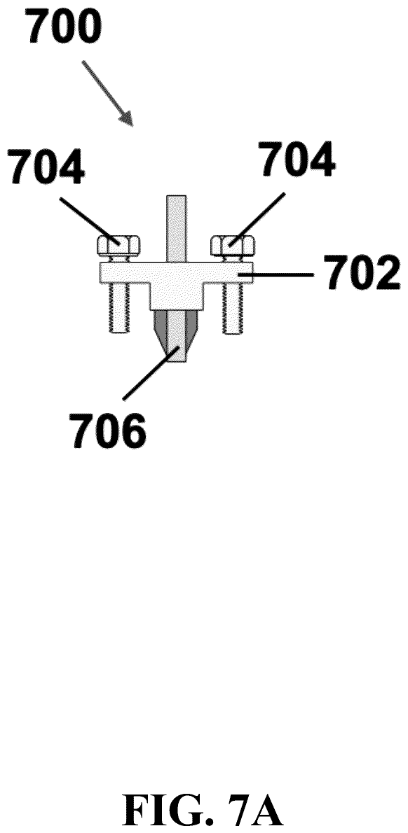

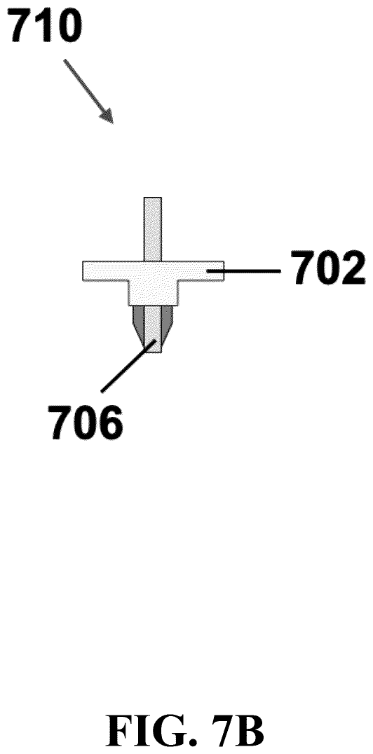

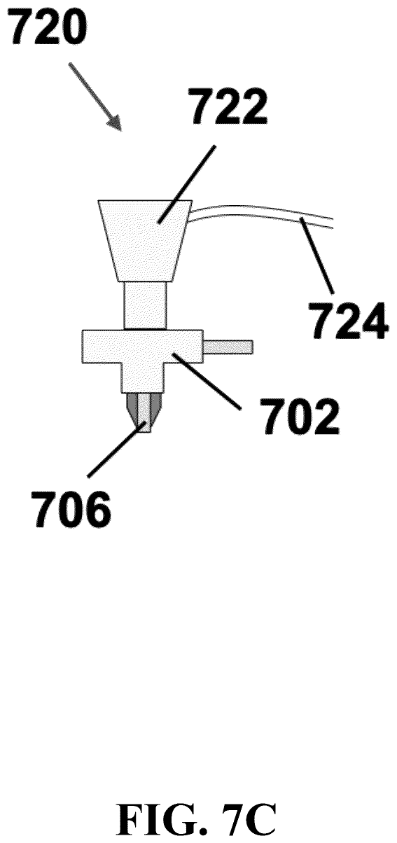

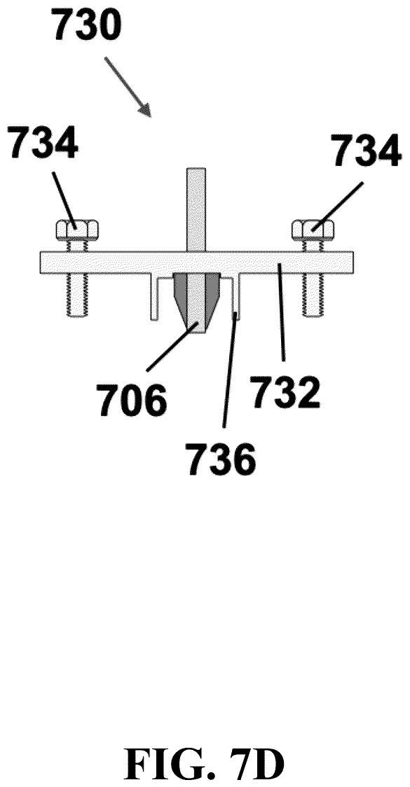

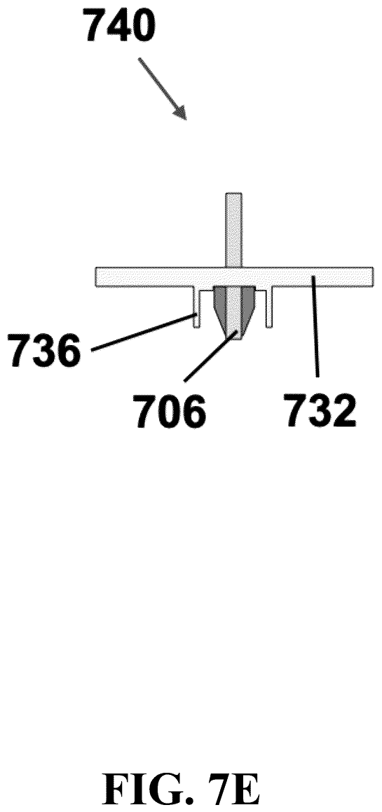

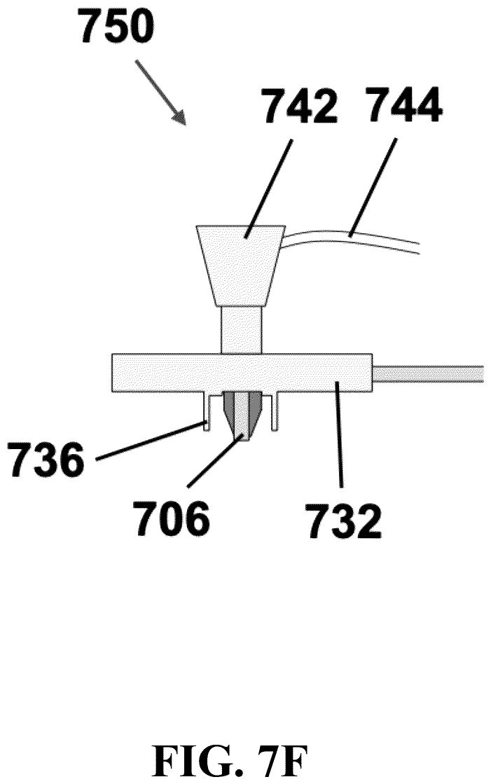

Similar to movable/rotatable chamber arrangements, immovable chamber arrangements can be made with fixed fixtures. In such fixtures, the fitting can be repositioned during each step of the process. FIGS. 61-6L show various chamber arrangements in a fixed strip. Specifically, FIG. 61 shows strip 610 having 3 chambers 612 disposed on the strip. Only one such chamber is labeled for the sake of convenience, but it should be appreciated that one or more such chambers 612 may be similar to, or the same as, any one or more other chambers described herein. Further, FIGS. 6J-6L show strip 610 having 4 chambers 612, 6 chambers 612, and 8 chambers 612, respectively. In several embodiments described herein, one or more chambers contain various elements and/or aspects that accommodate general plumbing fitting. Further, specific inlet fittings can be used, at least one embodiment of which is shown in FIG. 7A. Specifically, fitting 700 is a flange-type fitting that comprises flange 702, screws 704 to tighten the fitting, along with a ferrule and tubing 706. In at least another embodiment, FIG. 7B shows fitting 710 with a flange 702 and ferrule and tubing 706, but without the screws. In other embodiments, the flange can be secured using an active (powered) or passive mechanism e.g. hydraulic arm, spring mechanism, pneumatic slides as shown in FIG. 7C, allowing for easier connection and disconnection of the fitting in the aforementioned embodiments. Specifically, FIG. 7C shows fitting 720 with a flange 702, ferrule and tubing 706, and hydraulic arm 722 connected to tubing 724. The tubing can be connected to one or more portions and/or aspects of the apparatus (e.g., apparatus 100), such as, for instance, one or more pumps as described herein. In some embodiments, alignment features can be added to the fittings to help alleviate stress from fluidic connection parts, such as the ferrule and pilot. As a non-limiting example, FIG. 7D depicts a fitting 730 with a flange 732 with screws 734, threads, and an alignment feature 736. FIG. 7E shows a fitting 740 with a flange 732 and an alignment feature 736, but without screws 734. FIG. 7F shows a fitting 750 with a flange 732, alignment feature 736, and hydraulic attached fitting 742 with attached tubing 744 and an enclosing tongue to guide the fitting positioning.

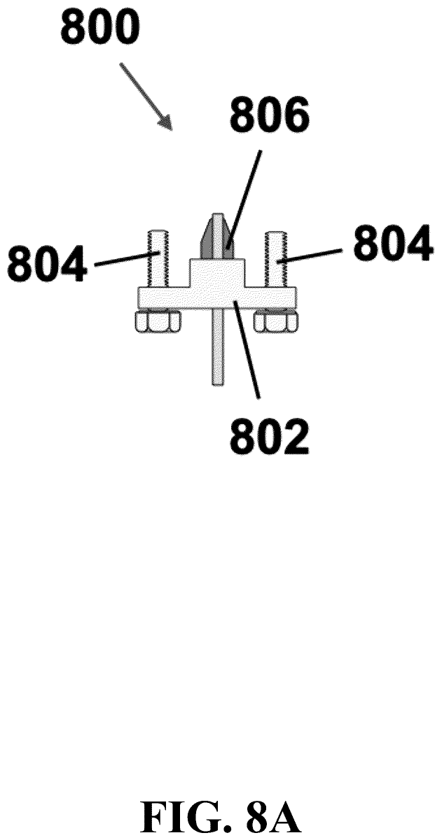

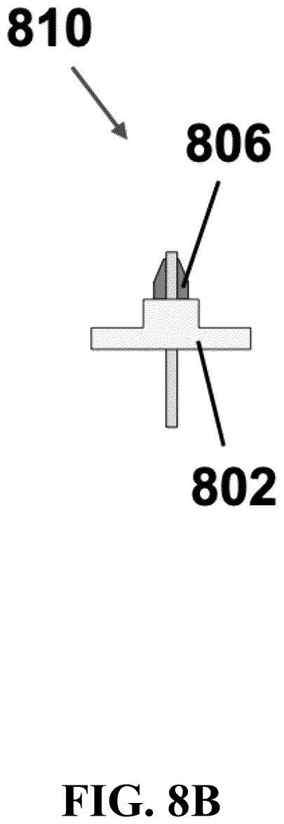

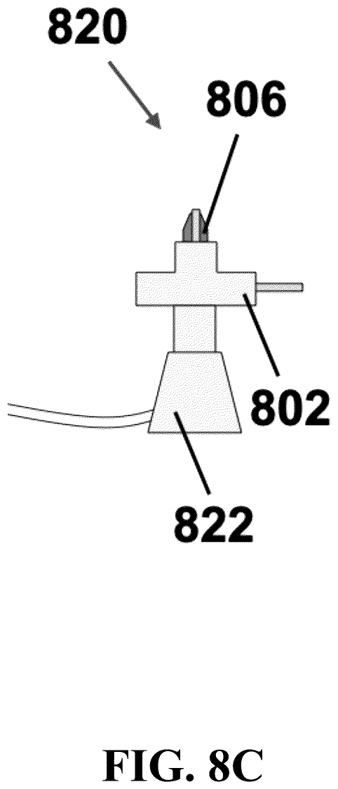

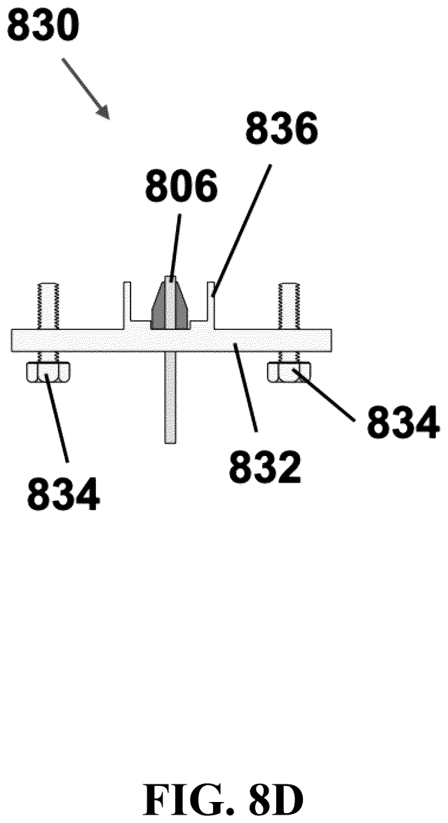

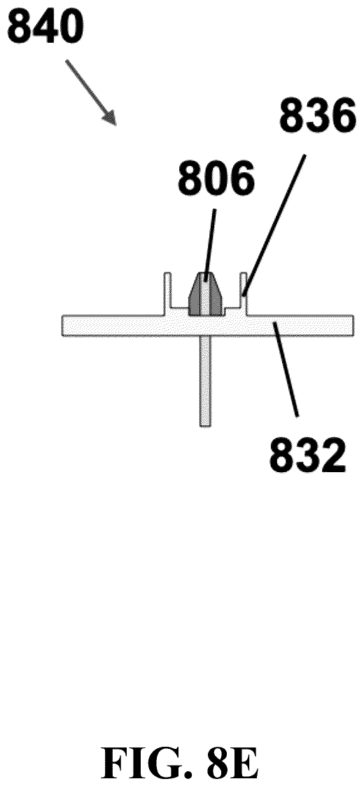

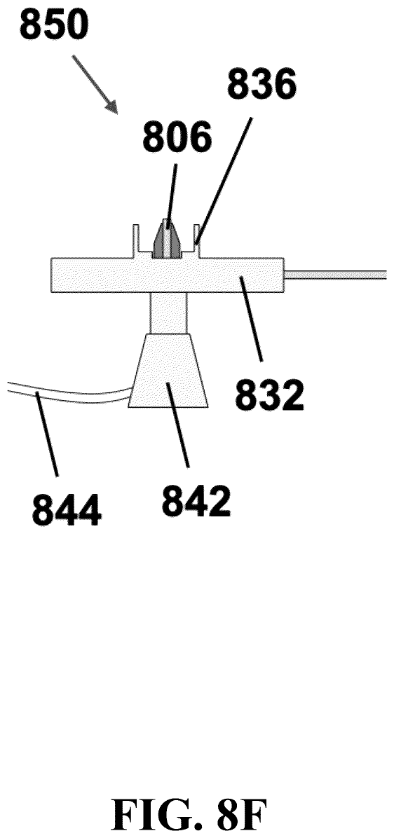

In at least one embodiment, the outlet fittings can be similar to, or the same as, the inlet fittings. For instance, the outlet fittings can have a similar, or identical, structure to the inlet fittings, and/or similar or identical portions and/or aspects. FIG. 8A illustrates fitting 800 suitable for certain embodiments, which is a flange-type fitting. The fitting 800 comprises flange 802, screws 804 for tightening, in addition to a ferrule and tubing 806. FIG. 8B shows fitting 810 with a flange 802 and ferrule and tubing 806, albeit without screws. In alternative configurations and/or embodiments, the flange may be secured using a hydraulic arm, as depicted in FIG. 8C. Specifically, fitting 820 has a flange 802, ferrule and tubing 806, and hydraulic arm 822, with associated tubing 824. As with embodiments of the fittings shown in FIG. 7C and FIG. 7F, the hydraulic arm shown in FIG. 8C may facilitate a more convenient engagement and disengagement of the fitting. Some embodiments may incorporate alignment features in the fittings to mitigate stress on fluidic connection components, like the ferrule and pilot. As a non-limiting example, FIG. 8D shows fitting 830 with a flange 832, screws 834, and alignment feature 836. FIG. 8E shows fitting 840 with a flange 832 and alignment features attachment 836, but without screws 834. Finally, FIG. 8F shows a flanged hydraulic-attached fitting with alignment features, specifically, fitting 850 with a flange 832, alignment feature 836, hydraulic arm 842, and associated tubing 844.

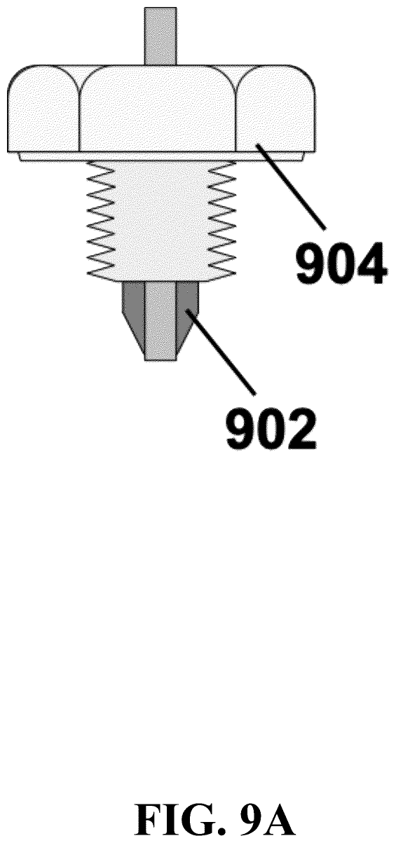

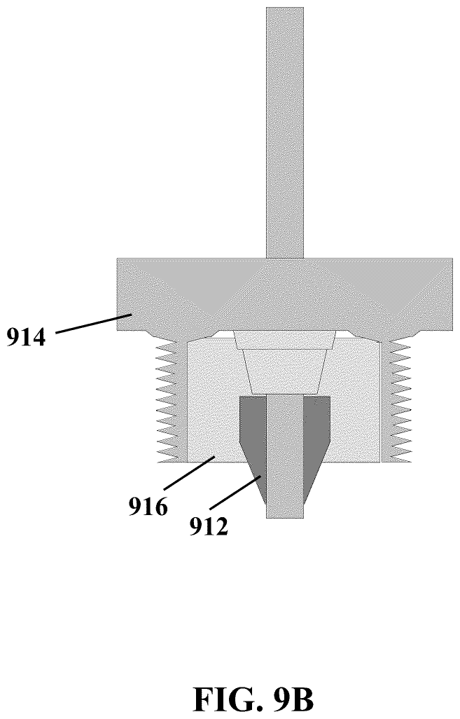

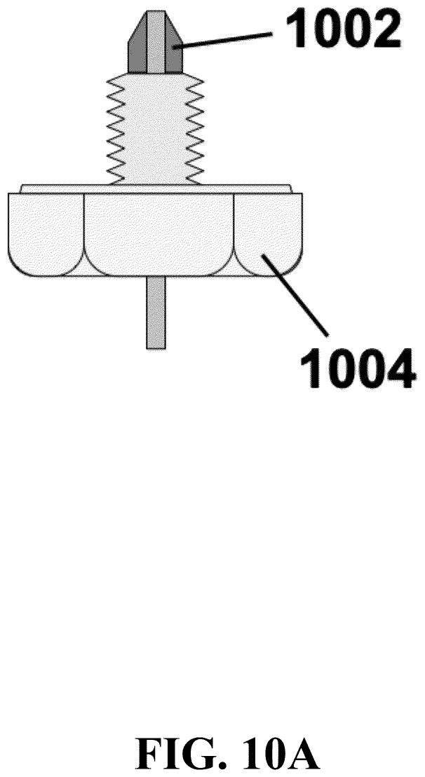

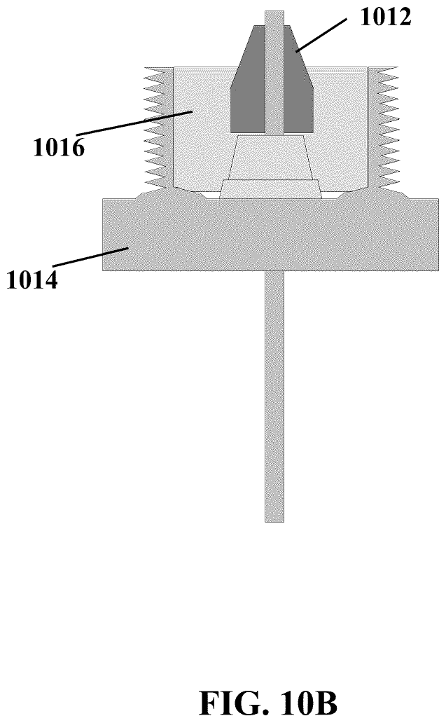

In embodiments having threaded-type chamber ends, nut-type inlets/inlet fittings can be configured in at least two ways: the ferrule can be placed directly below the nut, as shown in FIG. 9A, or inside the hollow nut to guide the ferrule and tube, as depicted in FIG. 9B. Specifically, FIG. 9A shows ferrule 902 disposed directly below nut 904, while FIG. 9B shows ferrule 912 disposed within nut 914, and specifically, one or more hollow spaces 916 within nut 914. FIGS. 10A-10B show embodiments of nut-type outlets/outlet fittings. Similar to FIG. 9A, FIG. 10A depicts a non-limiting example of a nut and ferrule-type connector having ferrule 1002 disposed directly above nut 1004. Similar to FIG. 9B, FIG. 10B depicts ferrule 1012 disposed within nut 1014, and specifically, one or more hollow spaces 1016 within nut 1014.

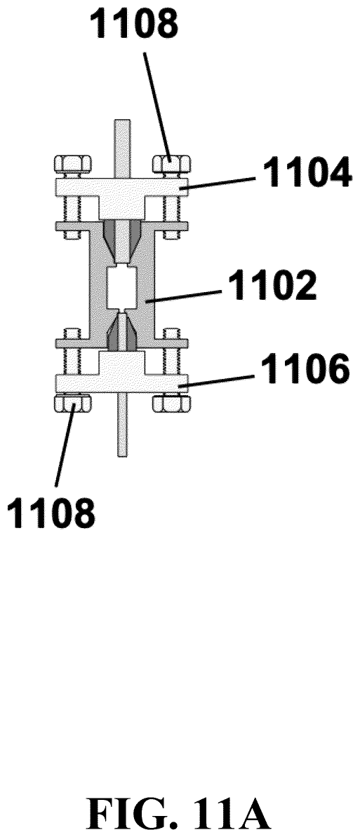

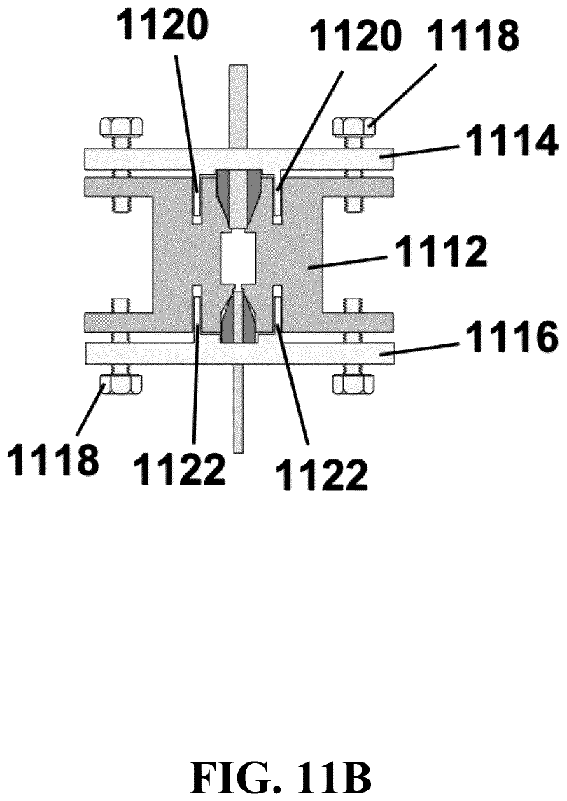

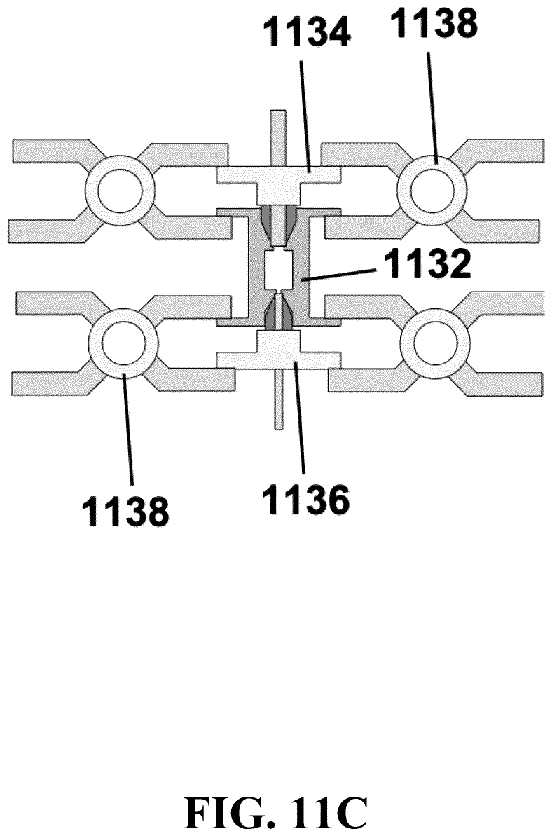

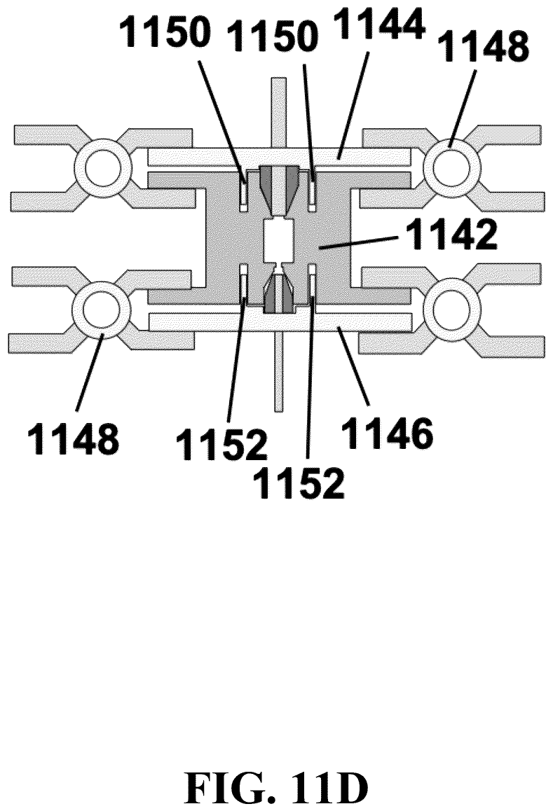

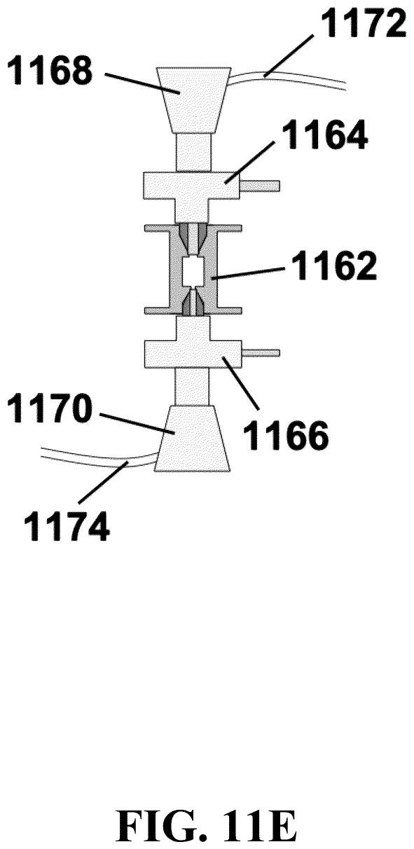

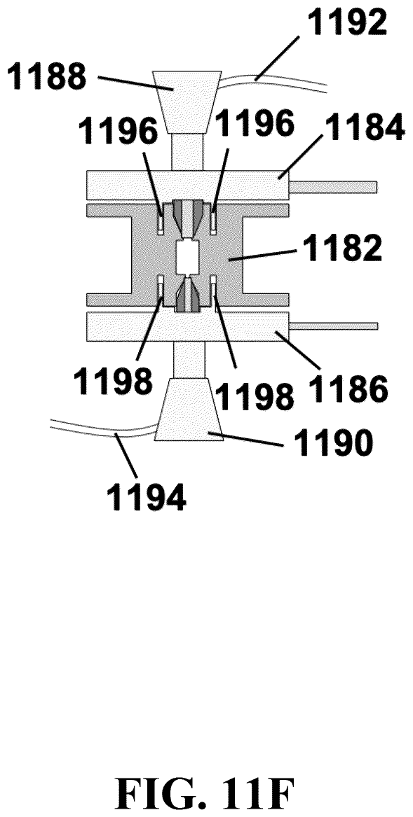

In at least one embodiment, a key requirement is proper alignment and/or engaging fittings to the chambers. The positioning and engaging of the inlet and outlet fitting to the chamber can be realized through several mechanisms, which depend on the type of fittings used. In the non-limiting example of screw-type connectors as described herein, FIG. 11A shows chamber 1102 connected with both the inlet fitting 1104 and the outlet fitting 1106. In at least one example, the inlet fitting may be similar to, or the same as, the outlet fitting. The aforementioned connections and/or connectors to the inlet and/or outlet fitting may be achieved through a flange and screw-type connector, including, for instance, screws 1108, of which only two are labeled for the sake of convenience. In at least one example, inlet fitting 1104 may be similar to, or the same as, fitting 700, while outlet fitting 1106 may be similar to, or the same as, fitting 800. In FIG. 11B, the connection is supported by additional alignment features, as described above herein. Chamber 1112 is attached to both inlet fitting 1114 and outlet fitting 1116. Also shown are screws 1118, of which only two are labeled for the sake of convenience, and alignment features 1120 and 1122. FIG. 11C depicts a chamber 1132 connected with inlet fitting 1134 and outlet fitting 1136, which are each secured through clamps 1138, only two of which are labeled for the sake of convenience. FIG. 11D shows a similar connection as in FIG. 11C, except with the addition of alignment features. Specifically, chamber 1142 is connected with inlet fitting 1144 and outlet fitting 1146, which are each secured through clamps 1148, only two of which are labeled for the sake of convenience. Further, alignment features 1150 and 1152 are shown. In some embodiments, the fittings can be secured through hydraulic or mechanical shafts, as depicted in FIG. 11E, or through a hydraulic and mechanical shaft along with a guide, as in FIG. 11F. Specifically, FIG. 11E shows chamber 1162, which is connected with inlet fitting 1164 and outlet fitting 1166. Both the inlet fitting and outlet fitting are connected to hydraulic or mechanical shafts 1168 and 1170, respectively. Attached tubing 1172 and 1174 is also shown. FIG. 11F shows chamber 1182, which is connected with inlet fitting 1184 and outlet fitting 1186. Both the inlet fitting and outlet fitting are connected to hydraulic or mechanical shafts 1188 and 1190, respectively. Attached tubing 1192 and 1194 is also shown. The guides/alignment features are shown at 1196 and 1198, respectively.

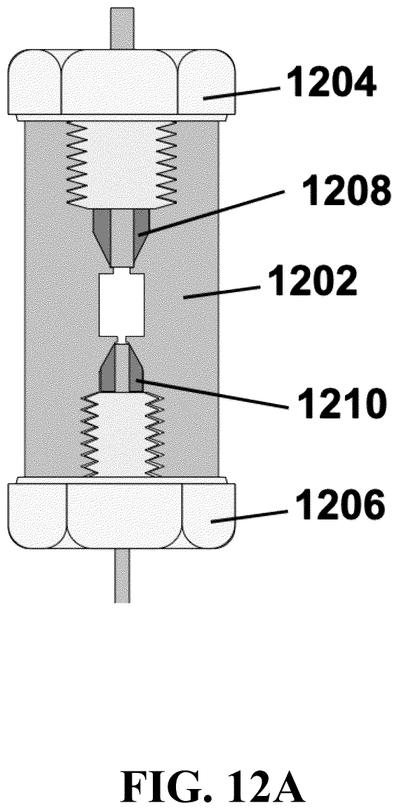

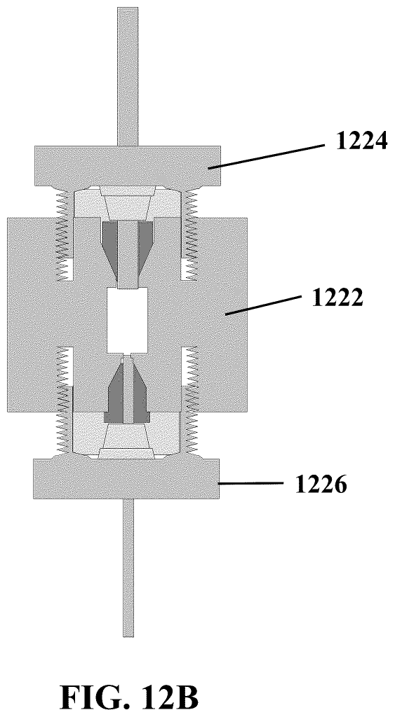

In at least one embodiment, connections through nut-type connections and/or connectors are depicted in FIGS. 12A-12B. Specifically, FIG. 12A shows chamber 1202 connected through nut-type connections and/or connectors having nuts 1204 and 1206, respectively. Each such nut has an associated ferrule and tube 1208 and 1210, respectively. It should be appreciated that the connections and/or connectors shown in FIG. 12A may be similar to, or the same as, the connections and/or connectors shown in FIG. 9A and FIG. 10A. FIG. 12B shows chamber 1222 connected through nut-type connections and/or connectors having nuts 1224 and 1226, respectively. Each such nut has an associated ferrule and tube 1228 and 1230, respectively. It should be appreciated that the connections and/or connectors shown in FIG. 12B may be similar to, or the same as, the connections and/or connectors shown in FIG. 9B and FIG. 10B.

### Elution of Analytes

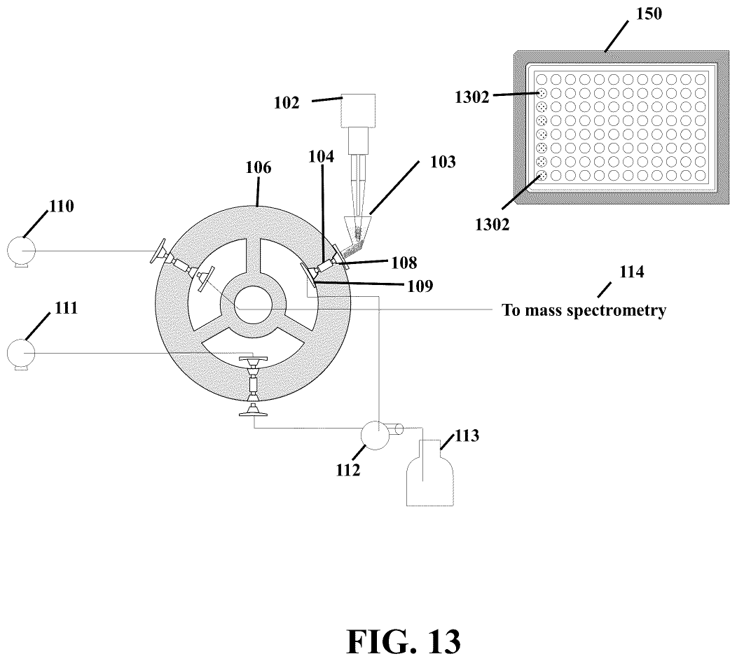

FIG. 13 describes, in at least one embodiment, the first step in the continuous and/or automated elution of analytes from one or more beads. In at least one example, eluted analytes may be analyzed via mass spectrometry analysis, as described herein. It should be appreciated that, although FIG. 13 shows the apparatus of FIG. 1, the continuous and/or automated elution of analytes is not limited to any specific one or more embodiments described herein. Beads, which may be reversibly conjugated with one or more analytes of interest, are stored in one or more buffers in the storage. As described herein, the storage may comprise a multi-well plate 150 that contains the sample of interest 1302, which may comprise one or more beads bound to one or more analytes. The bead manipulator 102 may move sample 1302 from the multi-well plate 150 to the bead feeder 103. As a non-limiting example, the bead manipulator may aspirate the sample using any one or more known methods, including, for instance, pipetting the sample into one or more pipette tips and/or storage areas within the bead manipulator 1302, and then ejecting the sample into the bead feeder 103.

One or more types of beads may be used with any one or more embodiments described herein, including, but not limited to, at least one embodiment shown in FIG. 13. Beads, sometimes called microspheres, may, in at least one example, typically between 1 micron and 1 mm in size. One or more types of beads may be made up of various materials such as polystyrene, polyethylene glycol (PEG), agarose, magnetic particles (e.g., iron oxide, nickel), silica, gold, polyacrylamide, polymethyl methacrylate (PMMA), polyvinyl alcohol (PVA), poly(lactic-co-glycolic acid) (PLGA), polyurethane, cellulose, chitosan, alginate, calcium carbonate, nylon, dextran, hydroxyapatite, zirconium oxide, titanium dioxide, zinc oxide, quantum dots, liposomes, ceramic, biodegradable polymers (e.g., poly(lactic acid), poly(glycolic acid)), and copolymers (e.g., polyethyleneimine-co-polyethylene glycol). In some embodiments, magnetic particles can be used, and such particles may be coated with other substances to derive functional surfaces that can attach to one or more analytes (e.g., via chemical bonding). In at least one embodiment, spherical particles are used. In at least another embodiment, non-spherical particles such as carbon micro/nanotubes or rods are also used interchangeably with beads. In at least one embodiment, beads may be treated to activate one or more surfaces on the beads in order to attach required molecules (e.g., one or more analytes). Chemical activation may be achieved with, for instance, epoxide compounds, carbodiimide reagents (e.g., EDC/NHS), glutaraldehyde, Traut's reagent (2-iminothiolane) to form an epoxy moiety, activated carboxyl groups, activated amino groups, and/or thiol groups. These activated groups may be used to attach biomolecules onto the surfaces. In at least one embodiment, commonly activated beads are conjugated with affinity reagents including, but not limited to, antibodies specific to protein analytes, antibody derivatives, protein complexes, enzymes, proteins/peptides, polynucleotides such as aptamers and/or any type of nucleic acid, fatty acids, and/or any other type of biological and/or chemical compound of interest.

In an embodiment, prior to the storage at the apparatus (e.g., in multi-well plate 150), the activated affinity beads may be pre-reacted with one or more samples 1302. Such samples may comprise biological samples or extract from biological samples. As a non-limiting example, biological fluids could include any one or more fluids extracted or originally derived from one or more organisms (e.g., blood, plasma, serum, urine, saliva, Cerebrospinal fluid (CSF), and the like). The samples may be pretreated to release the analytes in order to, for instance, remove some of the interfering molecules, enrich the molecules, or modify the analyte to enrich the capture on the beads. Some examples of sample processing may include extraction of proteins and lipid from proteoliposome complexes, removal of found proteins from nucleic acid (e.g., RNA and DNA) sequences, denaturation of proteins to release the linear epitopes, reduction and alkylation to break disulfide linkages, and proteolytic cleavage of proteins and digestion of nucleic acids (e.g., DNA or RNA). In some cases, the sample can also be treated with tags to label the samples (e.g., tandem mass tags). The reaction of the beads with the sample may also include addition of other chemical compounds to reduce non-specific binding. In at least one example, a high concentration of salt is used to void interactions due to ionic attraction. Further, one or more methods for analyte enrichment on beads are known in the art and well-described in various publications. In at least one embodiment, beads may be typically incubated in one or more samples (e.g., biological samples). The beads may then be washed to remove and/or weaken any non-specific binding. Washing may include several repeated washes with specific buffers, including, for instance, different types of buffers. The exact washing procedure and the buffer solution(s) used may be determined by the sample and the analyte of interest. More non-limiting examples of analyte elution methods are explained in the examples section below herein.

In at least one embodiment, one biological sample can be incubated with a plurality of beads. Each bead will contain affinity sites for an analyte present in the sample. These analytes can be a protein, protein complex, fragment of a protein, peptide, a metabolite, a lipid, a carbohydrate, a DNA fragment, mRNA or a mRNA fragment, or combination of these analytes. The number of beads may be determined by the number of analytes required to be analyzed. These beads are incubated with a pre-defined amount of biological samples (e.g., an amount necessary to produce the amount of analyte to be analyzed). Further, the amount of biological samples may be determined by the amount of analytes present in the sample. For example, if a set of analytes is measured from plasma, each of 100 beads that has an affinity for one analyte can be incubated with 50 microliters of plasma. Additionally or alternatively, 100 beads can be incubated in 100 microliters of plasma. In at least another example, 100 beads can be incubated with 150 microliters of plasma. Additionally or alternatively, 100 beads can be incubated in 200 microliters of plasma. Additionally or alternatively, 100 beads can be incubated in 500 microliters plasma. If the number of analytes is larger, the number of beads can be increased accordingly. For instance, 1000 beads may be incubated in 50 microliters of plasma. Additionally or alternatively, 1000 beads can be incubated in 100 microliters of plasma. Additionally or alternatively, 1000 beads can be incubated in 150 microliters of plasma. Additionally or alternatively, 1000 beads can be incubated in 200 microliters of plasma. Additionally or alternatively, 1000 beads can be incubated in 500 microliters of plasma.

Reaction of the beads with the samples (e.g., biological samples) can result in, and ensure, the capture of the analytes onto the beads. As described above herein, the beads can then be washed thoroughly to remove any non-specific binding. After washing, the washed beads can then be placed in a storage container and, specifically, multi-well plates as described above herein.

Continuous Processing and/or Elution of Analytes

In at least one embodiment, the process of continuous high-throughput elution of analytes begins with placing the pre-incubated bead sets in the bead storage area of the apparatus (e.g., multi-well plate 150 in apparatus 100). Upon receiving instructions from a control system and/or computer, the bead manipulator (e.g., manipulator 102) is positioned precisely at the bead storage location. Subsequently, the bead manipulator is lowered close to the wells/vials. The exact distance of the bead manipulator's descent is determined by various parameters, such as the fluid level in the well or vial, bead size, and suction or magnetic power of the bead manipulator. Once the manipulator reaches the required distance, the bead picking mechanism is activated. This mechanism may be embedded in, or otherwise comprised in, the bead manipulator, and may involve, for instance, lowering a paramagnetic rod inside the tip of the bead manipulator, magnetizing the rod already present in the tip of the bead manipulator, or activating the suction in a suction-type bead manipulator. Other methods of intaking and/or aspirating one or more samples may be used. After ensuring the picking of all beads in a single well, the bead manipulator is moved and positioned close to the bead feeder (e.g., bead feeder 103), engaged to the chamber (e.g., chamber 104) at the bead loading location.

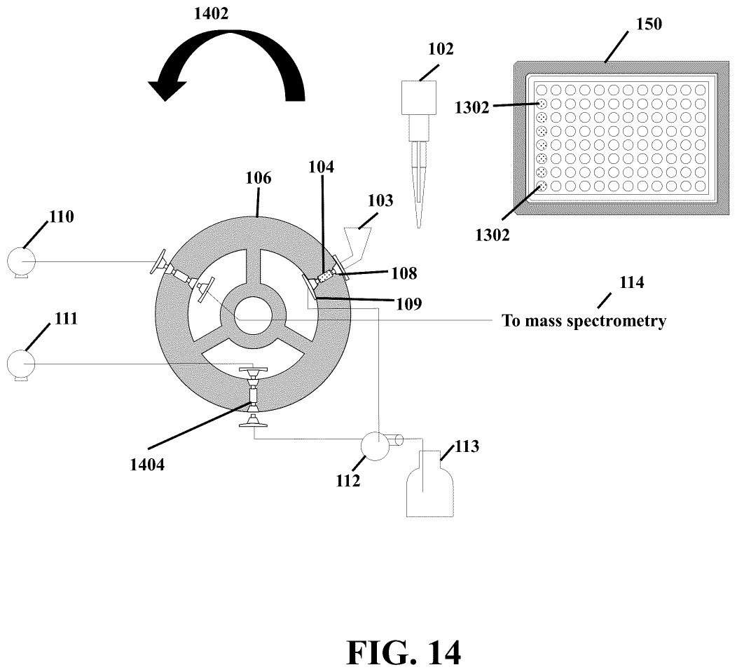

The chamber at the loading position is engaged with the bead feeder at the inlet (e.g., inlet fitting 108) and one of the outlet fittings (e.g., outlet fitting 109) at the outlet. The bead picking mechanism is then released to transfer the beads to the bead feeder. To facilitate bead transfer, the bead feeder may be sprayed with a wash buffer. This ensures the beads' transfer to chamber 104, as further shown in FIG. 14. Specifically, FIG. 14 illustrates the apparatus of FIG. 1 in a state in which beads fill one chamber 104. Any additional buffer filled into the chamber will be removed through the outlet fitting 109 and the vacuum pump connected to it (e.g., pump 112). In some embodiments, the chamber filled in this step may already be pre-filled with the wash solvent. In such arrangements, denser beads are placed into the lower positioned chamber by gravitational force alone. However, in such cases, the contact between the bead feeder and the chamber opening can be ensured by the vacuum arrangement, which removes any air bubbles that could prevent bead movement into the chamber. In some embodiments, the bead manipulator and the bead feeder may be connected as an integrated assembly, which can be engaged and disengaged with the chamber 10. In such configurations, the manipulator may comprise a suction head that could be connected to a fluid transfer system such as a pump.

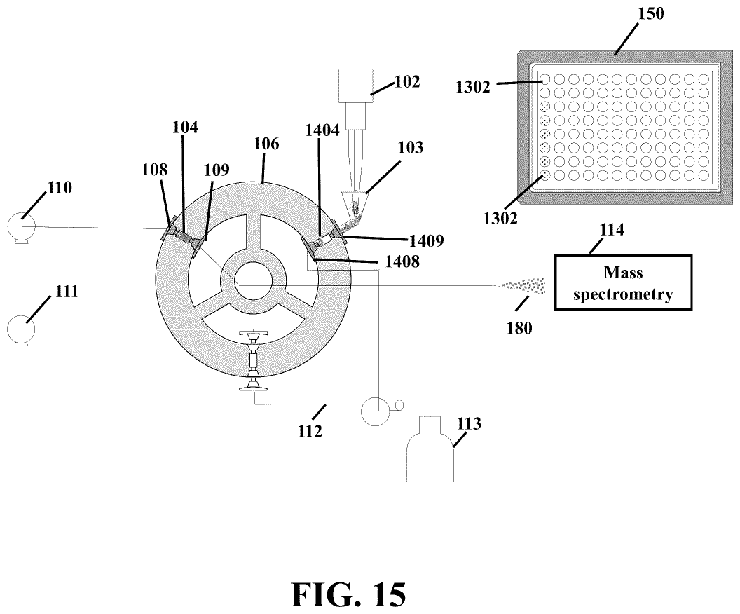

Once one chamber is filled with the appropriate bead set, the fittings and bead feeder are disengaged from the wheel 106, and the wheel rotates one-third of a turn (e.g., in an anti-clockwise direction 1402). This positions another chamber 1404 to the feeding location or closer to the feeder. Simultaneously, the chamber 104 that is already filled with beads reaches the elution position. The elution position is where the fittings are connected to the pump 110 containing the elution solvent and the outlet fitting connected to the external analytical instrument (e.g., mass spectrometry apparatus 114). This position is further shown in FIG. 15. Specifically, chamber 1404, with inlet fitting 1408 and outlet fitting 1409, is now in the position of chamber 104 as previously shown in FIGS. 13-14. That is, chamber 1404 is in a position to receive beads from the bead feeder 103.

As previously described with respect to chamber 104, bead manipulator 102 may transfer beads from a sample (e.g., sample 1302) to bead feeder 103, whereupon the beads are deposited into chamber 1404 (e.g., via inlet fitting 1408). Meanwhile, chamber 104 is now in a position to be connected to pump 110 containing the elution solvent (e.g., via inlet fitting 108). The outlet fitting 109 can be connected to an external analytical instrument, such as mass spectrometry apparatus 114.

Upon reaching the elution position, the fittings 108 and 109 are engaged, and the elution solvent is allowed to flow through the chamber 104 containing the beads. The composition of the elution solvent can be determined by the type of interaction between the one or more analytes of interest and the one or more beads employed in the apparatus. Thus, the composition of the elution solvent can be optimized to maximize elution efficiency. For example, if the interaction is an affinity between an antibody and antigen, the elution solvent may be a low pH or acidic solution, such as water with 0.1% formic acid, water with 0.1% acetic acid, a solution of 59.9% water and 40% acetonitrile with 0.1% formic acid, a solution of 59.9% water and 40% acetonitrile with 0.1% acetic acid, and the like. The elution solution is allowed to flow for a specific, pre-determined duration to ensure complete elution of analytes from the beads. The analytes eluted from the beads are released into the elution solvent stream, similar to a liquid chromatography system, and connected to the analysis system. Eluted analytes 180 may pass to, for instance, mass spectrometry apparatus 114.

While the first chamber undergoes the elution of analyte process, the second chamber 1404, positioned at the filling location, is filled with the beads programmed for analysis in the second cycle. The filling process for the second chamber 1404 mirrors that of the first chamber 104.

The analysis method of at least one embodiment, as guiding described herein, can be similar to liquid chromatography in the sense that analytes are eluted in a peak shape. In other words, once the elution buffer contacts the beads containing the analytes, such one or more analytes are displaced from the beads, gradually increasing over time and decreasing after reaching the peak, resulting in a binomial distribution elution profile. This elution process may occur for all beads inside any one or more chambers. The onset and end of elution depends on the initial contact between the bead and the elution solvent. This phenomenon may result in separate elution profiles or differences in elution time for each bead-analyte pair. This method enables the analysis of pre-bound beads, akin to LC-MS analysis.

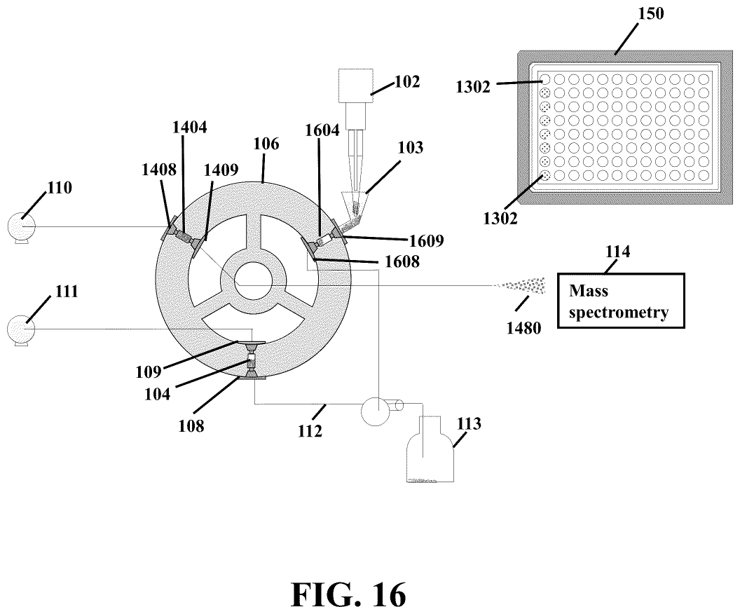

Once the elution process is completed at chamber wheel position 2 for chamber 104, that is, the position shown in FIG. 15, all fittings will be disengaged and the rotary wheel 106 will rotate another one-third rotation in the counterclockwise direction, as depicted in FIG. 16. Specifically, chamber 104, with associated fittings 108 and 149 is now at the third position. At the third position, the beads will be removed from the chamber. Here, it is preferable to have the chamber in the upright/down position (that is, a vertically-oriented or substantially vertically-oriented position) to enable gravity-based movement of the beads from the chamber. The flow of liquid through the outlet fitting and the second end of the chamber 104 will aid in the removal of the beads from the chamber, while the chamber is being removed at the third position. As shown, the third and final chamber 1604, with associated fittings 1608 and 1609, is now at the first position. That is, this chamber 1604 is now in a position to receive beads from the bead feeder 103. As previously described with respect to chamber 104 and chamber 1404, bead manipulator 102 may transfer beads from a sample (e.g., sample 1302) to bead feeder 103, whereupon the beads are deposited into chamber 1604 (e.g., via inlet fitting 1608). Meanwhile, chamber 1404 is now in a position to be connected to pump 110 containing the elution solvent (e.g., via inlet fitting 1408). The outlet fitting 1409 can be connected to an external analytical instrument, such as mass spectrometry apparatus 114. Thus, chamber 1604 will be filled with a new batch of bead sets, and the analytes 1480 associated with the beads in the second chamber (that is, chamber 1404) will be eluted at the second position. These analytes 1480 may then pass to an analytical instrument, such as mass spectrometry apparatus 114. This completes one full process cycle of one set of beads. After the removal of the beads, the chamber 104 will be ready to be filled with a new set of beads and undergo subsequent cycles, such as when the wheel 106 completes another one-third rotation anti-clockwise, bringing the chamber 104 back to its initial position shown in FIG. 13.

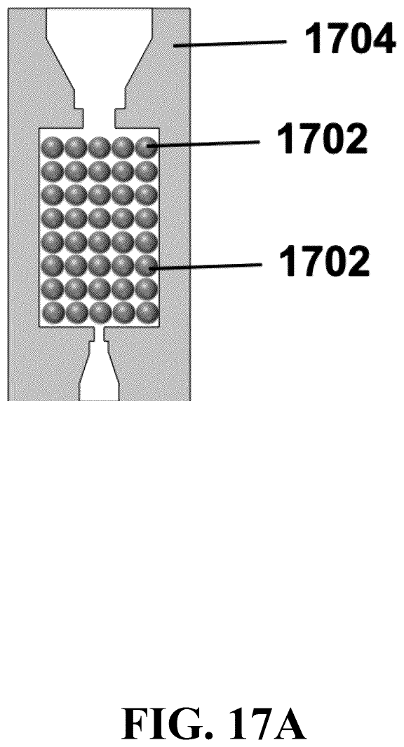

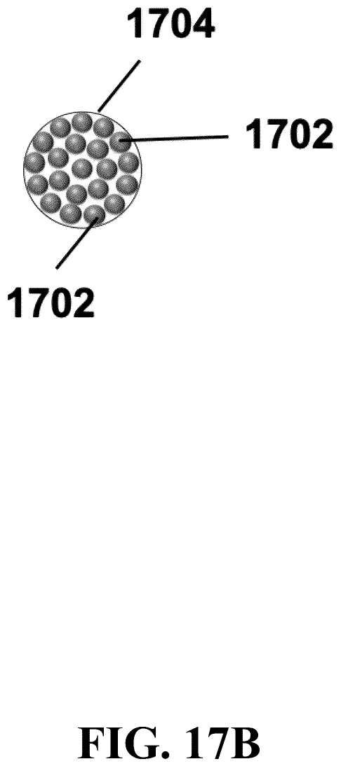

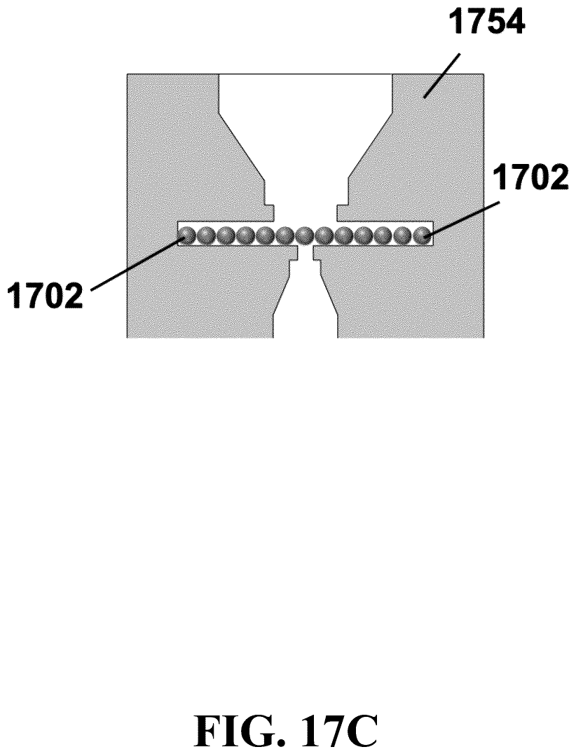

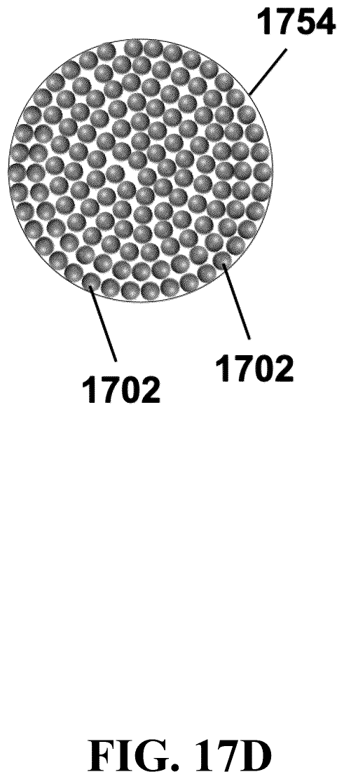

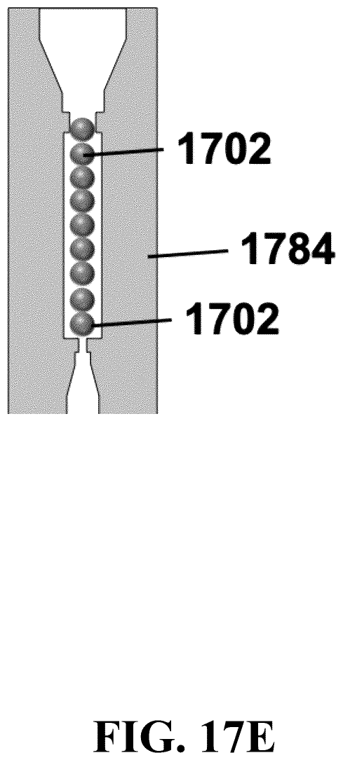

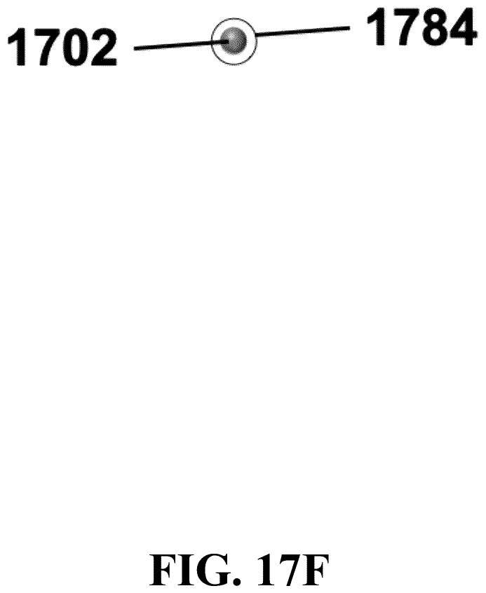

Embodiments of the invention disclosed herein describe an apparatus and method for high throughput analysis of multiplexed analytes captured from biological samples. The multiplexing capacity or number of analytes that can be analyzed simultaneously depends on the number of beads that can be packed at one time. For example, if 500 beads are packed at one time, 500 analytes can be analyzed from the sample. This number is derived based on the fact that one bead is used to capture one analyte. Similarly, 2000 beads can be used to analyze 2000 analytes simultaneously. The arrangement of a plurality of beads inside the chamber is determined by the dimensions of the chamber and the beads. FIGS. 17A and 17B depict the arrangement of beads 1702 inside chamber 1704, which may be similar to, or the same as, any of the chambers described herein). Only two such beads 1702 are labeled in each of FIGS. 17A-17B for the sake of convenience. As described previously, the length of one or more such chambers may be larger than the diameter of the chamber. Several layers of beads may be stacked on top of each other to form multiple layers, as depicted in FIG. 17A, which shows a side, cut-away view of the chamber. FIG. 17B is a top view of beads in the chamber; that is, looking down the length of the chamber.

FIGS. 17C and 17D depict the arrangement of a plurality of beads 1702 inside a different type of chamber 1754, in which the diameter of the chamber is much larger than its height. FIG. 17C shows a side, cut-away view of the chamber, while FIG. 17D is a top view of beads in the same chamber; that is, looking down the length of the chamber. Here, the height is designed to accommodate a single layer of beads. Again, only two such beads 1702 are labeled in each of FIGS. 17C-17D for the sake of convenience.

FIGS. 17E and 17F depict a bead arrangement of a single bead per layer inside another type of chamber 1784. This arrangement is possible by designing the diameter of the chamber between 1.1 times and 1.95 times the diameter of the beads. FIG. 17E is a side, cut-away view of the chamber showing beads 1702, while FIG. 17F is a top view of beads in the same chamber; that is, looking down the length of the chamber. Again, only two such beads 1702 are labeled in FIG. 17E for the sake of convenience. In FIG. 17F, only one bead 1702 is visible from a top-down view, since the beads are stacked one on top of each other. The design of the volume of chamber 1702, and/or any of the other chambers described herein, may depend upon one or more of the following parameters: (1) number of analytes to be analyzed, (2) size of the beads, (3) volume of the elution solvent required for complete elution of the analytes, (4) flow rate of the elution solvent, and (5) the separation power required to easily identify all of the analytes of interest. For example, if the analytes are separated enough in mass spectrometry, a single layer of beads as depicted in FIGS. 17C and 17D can be used, with a higher flow rate that may enable quicker analysis. At the same time, if the analytes are close enough in m/z and need separation on the time axis, a chamber that enables a single bead per layer arrangement, as depicted in FIGS. 17E and 17F, can be employed. Such a method can be accommodated with a lower flow rate to elute the samples one by one. The analytes associated with beads that come into contact with the elution solvent will be eluted and brought into the analysis regime first. This will be followed by the elution of the analytes associated with the beads in the second layer. Further, at least one embodiment described in FIGS. 17C and 17D can be used for higher multiplexing capacity, wherein the analytes have a range of m/z.

### High-Throughput Capabilities

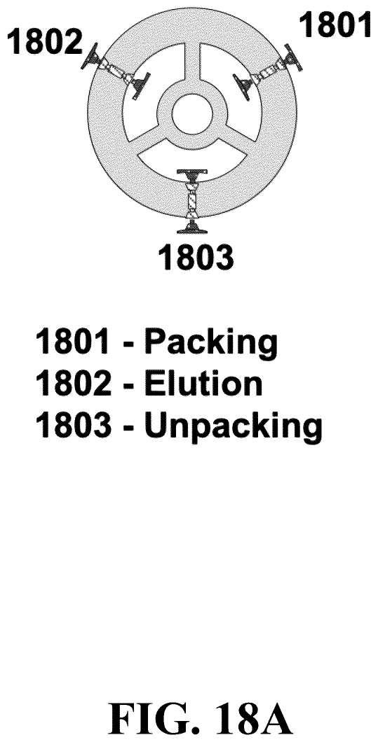

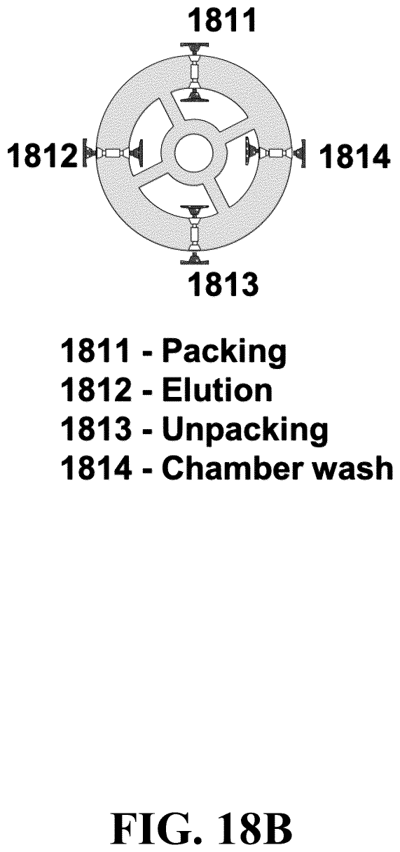

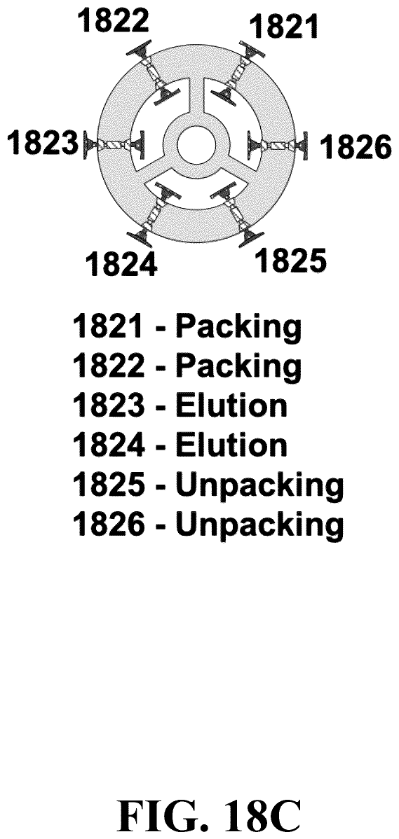

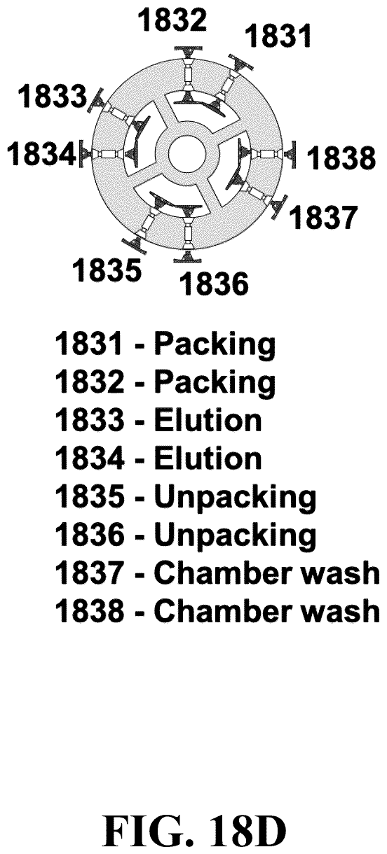

In at least one embodiment, three parameters—the volume of the chamber, the number of beads, and/or the flow rate of the elution buffer—will determine the high-throughput capacity of sample elution and/or analysis. Samples can be eluted using an amount of elution buffer equivalent to one chamber volume. For example, if a chamber measuring 1 mm×1 mm is filled with 100-micron beads and the flow rate of the elution buffer is 3.5 microliters per minute, it is possible to analyze 960 analytes per sample per minute. However, if there is a delay of 1 minute between each sample, the throughput would effectively be 960 analytes per 2 minutes per sample. This would enable the quantification of 960 analytes from 740 samples per day. If the size of the beads is reduced to 50 microns, then the number of analytes that can be quantified could increase to 7680 analytes from 740 samples per day. The details described previously herein represent the method usable for at least one embodiment wherein the number of identical chambers is 3 and the number of processing steps in each cycle is 3. In the case of an embodiment with more than 3 chambers, other parts of the apparatus can also be increased accordingly. For example, a system with 3 chambers will contain one bead manipulator, one bead feeder, 2 inlet fittings, and 3 outlet fittings. This system is associated with 3 functions at the various chambers, namely: (1) bead packing, (2) elution of analytes, and (3) bead unpacking. FIG. 18A depicts the various steps that can happen at different positions in the analysis device and/or system. Specifically, positions 1801, 1802, and 1803, are shown, with one chamber at each of the positions. As described above herein, each of these three positions on the wheel is associated with a specific function, specifically, packing at position 1801, elution at position 1802, and unpacking at position 1803. In certain embodiments, the system may contain a 4-chamber system enabling an addition of one more step in sample elution and/or analysis. As depicted in FIG. 18B, various positions 1811 through 1814 are shown. Packing of the beads in the chamber may occur at position 1811, elution of the analytes from the beads may occur at position 1812, and unpacking the beads from the chamber may occur at position 1813, and cleaning of the chamber after unpacking (and/or priming the chamber for the next cycle of analysis) may occur at position 1814. The addition of one or more positions and/or steps for cleaning of one or more chambers after unpacking, and/or priming the chamber for a further cycle of analysis, may have further benefits, such as additional reductions in cross-contamination of analytes from one set of samples to another set of samples. In a non-limiting example in which the analysis device and/or system comprises 4 chambers (e.g., as shown in FIG. 18B), the system may contain 1 bead manipulator, 1 bead feeder, 3 inlet fittings, and 4 outlet fittings.

In at least one embodiment, each of the one or more chambers (e.g., each of three chambers, four chambers, six chambers, eight chambers, or more than eight chambers) can be classified as one chamber for one round of analyte elution and/or processing. In some embodiments, multiple chambers can be used to perform one process. In such cases, one chamber can be engaged with the essential fittings and connected to the necessary pumps, while in another chamber, the packing and elution steps can happen. FIG. 18C shows an analysis device and/or system with 6 chambers and various positions for each chamber. In this system, 2 chambers will be used for packing, 2 chambers for elution, and 2 chambers for bead unpacking. Accordingly, the system will have 1 bead manipulator, 2 bead feeders, 4 inlet fittings, and 6 outlet fittings. As shown, there are six positions corresponding to each of the six chambers, with packing of the beads at positions 1821 and 1822, elution of the analytes from the beads at positions 1823 and 1824, and unpacking of the beads from the chamber at positions 1825 and 1826. Similar to the 6-chamber system, which is functionally similar to the 3-chamber system, an 8-chamber system may be used, which is similar to a 4-chamber system. As depicted in FIG. 18D, such a system will have 2 chambers for bead packing, 2 chambers for analyte elution, 2 chambers for unpacking of beads, and 2 columns for chamber wash. Accordingly, there would be 1 bead manipulator, 2 bead feeders, 6 inlet fittings, and 8 outlet fittings. The number of pumps can be retained the same or increased. In cases where the pumps are retained, each fitting will be connected with a 2-1 valve. Specifically, as shown in FIG. 18D, there are eight positions corresponding to each of the eight chambers, with packing of the beads at positions 1831 and 1832, elution of the analytes from the beads at positions 1833 and 1834, unpacking of the beads from the chamber at positions 1835 and 1836, and cleaning of the chamber after unpacking (and/or priming the chamber for the next cycle of analysis) at positions 1837 and 1838.

In these embodiments with 6 and 8 chambers, one chamber will undergo bead processing while the other chamber is engaged with the fittings, for example. For instance, the chamber at position 1821 will be attached to the bead feeder at one end of the chamber and an outlet fitting at the other end. At the same time, the chambers at positions 1823 and 1825 will be engaged with elution and unpacking fittings at both ends. Now, once the appropriate processing starts, the chamber at position 1821 will undergo bead packing, the chamber at position 1823 will undergo bead elution, and the chamber at position 1825 will undergo bead unpacking simultaneously. At the same time, the chambers at positions 1822, 1824, and 1826 will be engaged with the required chamber fittings. Once the processing in the chambers at positions 1821, 1823, and 1825 is completed, the valves will switch, and all processing will happen in the chambers at positions 1822, 1824, and 1826. By performing fitting engagement in parallel with bead processing, this embodiment can reduce the total analysis time compared to conventional systems utilizing three or four chambers.

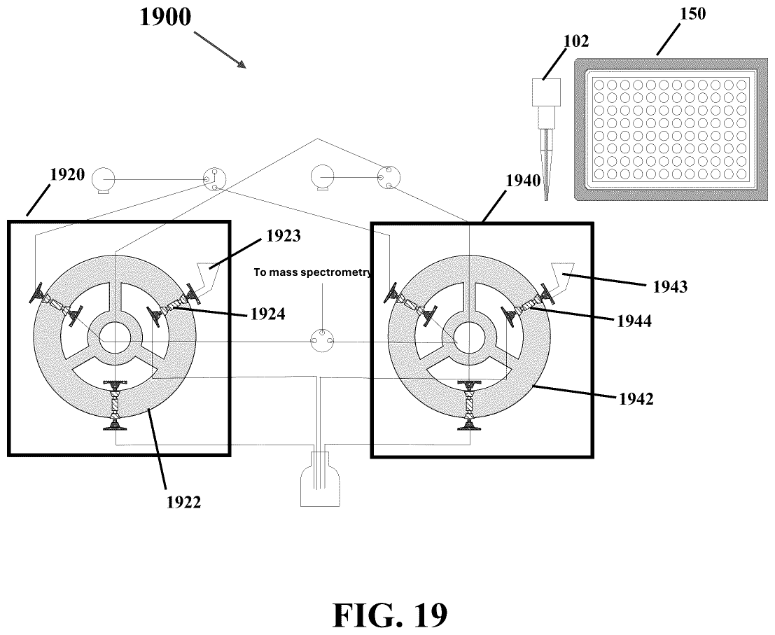

In at least one embodiment, the system's high-throughput capability can be significantly improved by eliminating the time required for engaging and disengaging fittings between steps and/or positions as the chambers move from one position to another. This can be achieved, in at least one example, using two identical sets of wheels or chamber arrangements, as depicted in FIG. 19. System 1900 is shown in which there is a multi-well plate 150 and a bead manipulator 102, as in previous embodiments described herein. However, there are two individual sets of equipment 1920 and 1940. Each set comprises one wheel (specifically, wheel 1922 and wheel 1942, respectively) and various chambers, only one of which is labeled for convenience (specifically, chambers 1924 and 1944, respectively). Further, each set has one bead feeder (specifically, feeder 1923 and 1943, respectively). In system 1900, one set undergoes bead filling, analyte elution, and bead unpacking, while the other undergoes engaging and disengaging. This eliminates the time taken for engaging and disengaging during each step. The system comprises one bead manipulator, two feeders, four inlet fittings, and twelve outlet fittings. It uses three pumps with 2-1 valves attached between each pump and the pair of fittings connected to the specific chamber.

At the start of sample elution and/or analysis in system 1900, a chamber in the first set 1920 (e.g., chamber 1924) is attached to the bead feeder (e.g., feeder 1923), and the bead manipulator 102 transfers the beads to the chamber. Simultaneously, a chamber in the second set 1940 (e.g., chamber 1944) is prepared for the packing step by engaging the bead feeder (e.g., feeder 1943) to it. After this, the chamber 1924 can be prepared for the elution step, as described above herein, while the chamber 1944 is packed with beads. The valve attached to the elution solvent pump is then opened to feed the chamber 1924, and the analytes associated with the beads are eluted. This occurs while the chamber 1944 is prepared for the elution step by engaging the input and output valves to the chamber.

After these steps, the elution valve is redirected to flow the elution buffer to the chamber 1944. Simultaneously, the chamber 1924, which has already been eluted, is prepared for the unpacking step. This includes disengaging the elution fittings, rotating the wheels by another one-third turn to bring the chamber to the unpacking position, and engaging the unpacking fittings. Once the elution and unpacking preparation steps are completed, the chamber 1944 is disconnected from the elution fittings, rotated by one third of a turn, and engaged with the unpacking fittings. At the same time, the chamber 1924 undergoes the unpacking procedure. Once this is done, the chamber 1924 is prepared for packing the new bead set, and the chamber 1944 is unpacked after redirecting the valve to the second wheel. This process is repeated until all bead sets in the bead storage have been processed.

### Various Features of Embodiments of the Invention

Various advantages of one or more embodiments of the invention disclosed herein are discussed below. It should be appreciated that such advantages are non-limiting, and more than one advantage may be present in any one or more embodiments.

One of the significant advantages of at least one embodiment of the present invention is the ability to facilitate high-throughput quantification of proteins, including those that have undergone post-translational modifications, using mass spectrometry. This process is a critical component of proteomics analysis, which is typically conducted at three distinct levels: top-down proteomics, bottom-up proteomics, and middle-down proteomics. In top-down proteomics, proteins are analyzed in their intact form. This approach allows for the comprehensive analysis of the protein, including its various modifications and isoforms, providing a holistic view of the protein's function and role within the biological system. Middle-down proteomics involves the analysis of peptide fragments that are endogenously generated within the biological samples. These fragments, which are larger than those used in bottom-up proteomics, provide a more detailed view of the protein's structure and modifications, offering insights into the protein's function and interactions within the cell. For bottom-up proteomics, the protein sample is digested using a proteolytic enzyme, typically trypsin, to produce a specific linear sequence of polypeptides. These polypeptides are then analyzed using mass spectrometry. This approach is particularly useful for identifying and quantifying individual proteins within complex mixtures, making it a powerful tool for large-scale proteome analysis. Each of these approaches offers unique advantages and potential challenges, and the choice of method depends on the specific goals of the proteomics analysis. Together, they provide a comprehensive toolkit for the study of proteins and their roles within biological systems. Embodiments of the invention therefore enhance the efficiency and throughput of these analyses, contributing to advancements in the field of proteomics. The apparatus and the method described in the disclosure offer a unique advantage of performing at all three levels of proteomics techniques in a single instrument.

Additionally, in at least one embodiment, the devices, systems, and/or methods described herein enable continuous, scalable analysis of multiplexed protein quantification using electron spray ionization techniques. There are no currently available technologies and/or apparatuses available for the quantification of proteins bound to multiplexed beads using electron spray mass spectrometry. The aforementioned at least one embodiment is advantageous over other methods such as those developed to array beads onto MALDI-plates and elute them on the plates. Such currently known methods require manual arrangement and/or arraying onto various slides (e.g., using a micro-well gasket). By contrast, at least one embodiment of the invention is partially and/or fully automated.

Further, at least one embodiment of the invention has one or more advantages compared to known methods that only elute beads in a position-specific manner. As described herein, analyte elution can occur in both a time- and position-dependent manner, providing at least one additional separation dimension.

Still further, at least one embodiment of the invention has one or more advantages compared to known methods that require eluting analytes on a plate (e.g., a MALDI-plate). As described herein, samples and/or analytes are eluted directly to an analysis apparatus, such as a mass spectrometry apparatus. Thus, at least one embodiment of the invention is not dependent on MALDI-TOF.

Still further, at least one embodiment of the invention has one or more advantages compared to known methods that cannot elute beads but only produce bead arrays. At least one embodiment is also faster at bead array development than currently known methods and/or approaches.

Still further, at least one embodiment of the invention has one or more advantages over non-mass spectrometry-based multiplexed bead assays, such as, for instance, Luminex's xMAP® technology, BD Biosciences' Cytometric Bead Array (CBA), and Quanterix's Single Molecule Array (SiMoA). In these methods, proteins are typically detected using a secondary signal such as fluorescence, which is associated with either the beads themselves or with secondary antibodies. By contrast, at least one embodiment of the invention utilizes a mass spectrometry-based quantification method. This technique not only matches the sensitivity of light-based detection methods, but also provides additional structural information about the proteins or peptides. The number of analytes that can be multiplexed in a single analysis by known methods is limited by the number of light channels the analyzer can detect without interference from other channels. In terms of mass spectrometry, the current high-resolution mass spectrometers can distinguish components with parts-per-million (PPM) level selectivity, indicating that they can analyze millions of masses simultaneously, thus providing the potential for virtually limitless multiplexing. Still further, at least one embodiment of the invention has one or more advantages compared to known methods that operate in a batch process (e.g., certain immune-affinity mass spectrometry assays), which requires several distinct steps. In such known methods, analytes are initially eluted from beads that have reacted with individual samples. This elution can be done either sequentially for each sample or for beads arranged in a 96-column format. After elution, the samples undergo LC-MS/MS analysis, which may or may not include additional sample processing steps such as concentration using a speed vacuum or lyophilization, and solid-phase extraction to remove impurities. These known methods face limitations in two critical areas: throughput and sensitivity. Even with short chromatographic runs, such as 2 minutes or 5 minutes, the necessary equilibration time for LC columns extends the total time required for analyzing each sample, thereby limiting the assay's throughput. Additionally, after the enrichment step, the analytes are often present in lower concentrations, which can lead to significant sample loss and reduce the sensitivity of the analysis.

At least one embodiment of the invention bypasses the liquid chromatography (LC) step entirely by eluting the analytes directly into the mass spectrometer in a manner similar to LC elution. This is achieved through, for instance, the apparatus and/or system described herein, which combines bead handling, multiple chambers, and a fluid flow system. Such an arrangement is functionally similar to, or the same as, an LC column on-site for each set of multiplexed beads. This arrangement allows for analytes to be eluted completely and read directly by the mass spectrometer without any losses due to sample handling steps. After elution, the elution aspects of at least one embodiment are discarded, and the apparatus and/or system is prepared for a new cycle of analysis with a new sample. This innovative feature of an essentially recyclable ad-hoc formation of an LC column is unique and enhances both scalability and throughput.

Embodiments of the present disclosure will be further understood by reference to the following non-limiting examples.

### EXAMPLES

It should be known by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### Example 1: Results Expected from Four Bead Sets

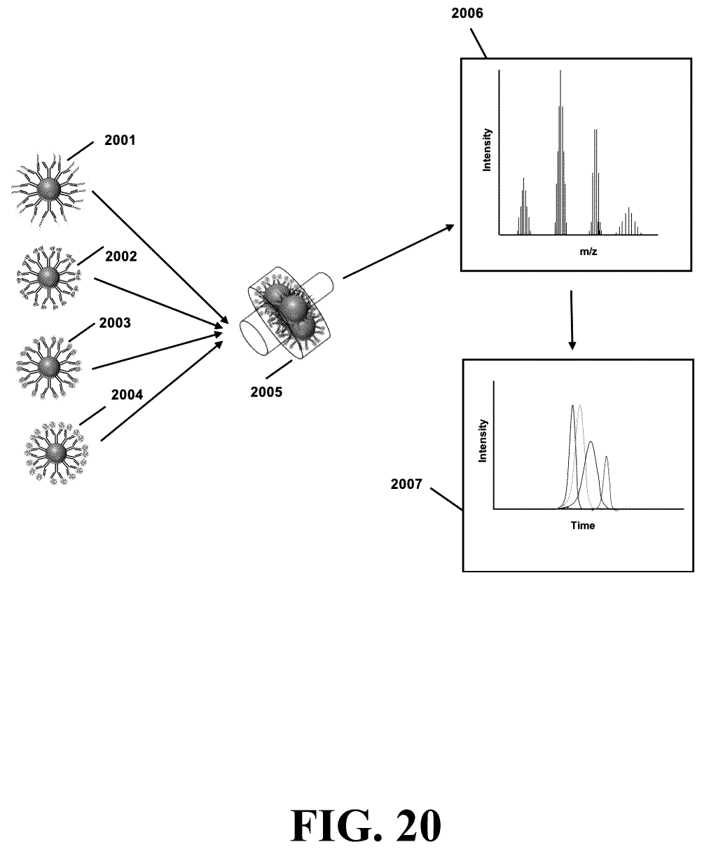

FIG. 20 shows an example of experimental results expected from a bead set of 4. Here, individual beads are attached to an affinity reagent (antibody). It should be appreciated that different affinity reagents may be used. The beads attached to the affinity reagents are then reacted with biological samples to form a reversible analyte-antibody conjugation. The biological samples may be pre-treated based on the type of proteome techniques and the type of sample. Table 1 below describes the potential non-limiting examples of methods that can be used for various biological samples:

TABLE 1
Types of biological samples, analysis types, and pre-treatment methods

| Biological | Type of | |
|---|---|---|
| sample | analysis | Potential pretreatment |
| Plasma/serum | Top down | Dilution - Addition of chaotropic agents such as urea, |
| | | guanidine hydrochloride - Delipidation - Reduction - |
| | | Addition of detergents - Addition of buffer - Addition of |
| | | salts - Alkylation-Dilution |
| | Middle down | Dilution - Addition of chaotropic agents such as urea, |
| | | guanidine hydrochloride - Delipidation - Reduction - |
| | | Addition of detergents - Addition of buffer - Addition of |
| | | salts - Alkylation-Dilution |
| | Bottom up | Dilution - Addition of chaotropic agents such as urea, |
| | | guanidine hydrochloride - Delipidation - Reduction - |
| | | Addition of detergents - Addition of buffer - Addition of |
| | | salts - Alkylation - Digestion with proteases-Dilution |
| Cerebrospinal | Top down | Addition of chaotropic agents such as urea, guanidine |
| fluid | | hydrochloride - Delipidation - Reduction - Addition of |
| | | detergents - Addition of buffer - Addition of salts - |
| | | Alkylation-Dilution |
| | Middle down | Addition of chaotropic agents such as urea, guanidine |
| | | hydrochloride - Delipidation - Reduction - Addition of |
| | | detergents - Addition of buffer - Addition of salts - |
| | | Alkylation-Dilution |
| | Bottom up | Addition of chaotropic agents such as urea, guanidine |
| | | hydrochloride - Delipidation - Reduction - Addition of |
| | | detergents - Addition of buffer - Addition of salts - |
| | | Alkylation - Digestion with proteases-Dilution |
| Saliva | Top down | Addition of chaotropic agents such as urea, guanidine |
| | | hydrochloride - Reduction - Addition of detergents - |
| | | Addition of buffer - Addition of salts - Alkylation- |
| | | Dilution |
| | Middle down | Addition of chaotropic agents such as urea, guanidine |
| | | hydrochloride - Reduction - Addition of detergents - |
| | | Addition of buffer - Addition of salts - Alkylation- |
| | | Dilution |
| | Bottom up | Addition of chaotropic agents such as urea, guanidine |
| | | hydrochloride - Reduction - Addition of detergents - |
| | | Addition of buffer - Addition of salts - Alkylation - |
| | | Digestion with proteases-Dilution |
| Urine | Top down | Addition of chaotropic agents such as urea, guanidine |
| | | hydrochloride - Reduction - Addition of detergents - |
| | | Addition of buffer - Addition of salts - Alkylation- |
| | | Dilution |
| | Middle down | Addition of chaotropic agents such as urea, guanidine |
| | | hydrochloride - Reduction - Addition of detergents - |
| | | Addition of buffer - Addition of salts - Alkylation- |
| | | Dilution |
| | Bottom up | Addition of chaotropic agents such as urea, guanidine |
| | | hydrochloride - Reduction - Addition of detergents - |
| | | Addition of buffer - Addition of salts - Alkylation - |
| | | Digestion with proteases-Dilution |
| Tissue | Top down | Tissue homogenization - Cell lysis using mechanical, |
| | | physical, chemical, enzymatic or osmotic method - |
| | | Removal of unlysed cell debris using centrifugation or |
| | | filtration - Addition of chaotropic agents such as urea, |
| | | guanidine hydrochloride - Reduction - Addition of |
| | | detergents - Addition of buffer - Addition of salts - |
| | | Alkylation-Dilution |
| | Middle down | Tissue homogenization - Cell lysis using mechanical, |
| | | physical, chemical, enzymatic or osmotic method - |
| | | Removal of unlysed cell debris using centrifugation or |
| | | filtration - Addition of chaotropic agents such as urea, |
| | | guanidine hydrochloride - Reduction - Addition of |
| | | detergents - Addition of buffer - Addition of salts - |
| | | Alkylation-Dilution |
| | Bottom up | Tissue homogenization - Cell lysis using mechanical, |
| | | physical, chemical, enzymatic or osmotic method - |
| | | Removal of unlysed cell debris using centrifugation or |
| | | filtration - Addition of chaotropic agents such as urea, |
| | | guanidine hydrochloride - Reduction - Addition of |
| | | detergents - Addition of buffer - Addition of salts - |
| | | Alkylation - Digestion with proteases-Dilution |
| Cell culture | Top down | Cell lysis using mechanical, physical, chemical, |
| | | enzymatic or osmotic method - Removal of unlysed cell |
| | | debris using centrifugation or filtration - Addition of |
| | | chaotropic agents such as urea, guanidine hydrochloride - |
| | | Reduction - Addition of detergents - Addition of buffer - |
| | | Addition of salts - Alkylation-Dilution |
| | Middle down | Cell lysis using mechanical, physical, chemical, |
| | | enzymatic or osmotic method - Removal of unlysed cell |
| | | debris using centrifugation or filtration - Addition of |
| | | chaotropic agents such as urea, guanidine hydrochloride - |
| | | Reduction - Addition of detergents - Addition of buffer - |
| | | Addition of salts - Alkylation-Dilution |
| | Bottom up | Cell lysis using mechanical, physical, chemical, |
| | | enzymatic or osmotic method - Removal of unlysed cell |
| | | debris using centrifugation or filtration - Addition of |
| | | chaotropic agents such as urea, guanidine hydrochloride - |
| | | Reduction - Addition of detergents - Addition of buffer - |
| | | Addition of salts - Alkylation - Digestion with proteases- |
| | | Dilution |

In the example depicted in FIG. 20, four beads labeled 2001, 2002, 2003, and 2004, each conjugated with a specific antibody, were reacted with a biological sample. These beads were packed in a chamber, and the eluted substance 2005, was subjected to mass spectrometry analysis. Any of the device and/or apparatus embodiments described herein may be used in the context of FIG. 20. Such device and/or apparatus embodiments may be integrated into any known one or more mass spectrometers. Such one or more spectrometers can perform mass spectrometry analysis utilizing the electron spray ionization method. Non-limiting examples of commercial mass spectrometers that can be used include, for instance, the Orbitrap series (e.g., Q Exactive, Orbitrap Fusion, Orbitrap Exploris), Triple quadrupole mass spectrometers (e.g., TSQ Altis, TSQ Quantis), Ion trap mass spectrometers (e.g., Velos Pro, LTQ XL) from Thermo Fisher Scientific; SYNAPT G2 series, Xevo series (e.g., Xevo TQ-XS, Xevo TQ-S), Vion series from Waters Corporation; Triple quadrupole mass spectrometers (e.g., Triple Quad 7500, Triple Quad 6500), QTRAP series, TripleTOF series (e.g., TripleTOF 6600, TripleTOF 5600) from SCIEX; timsTOF series, maXis series, from Bruker Corporation; 6400 Series Triple Quadrupole LC/MS, 6500 Series Accurate-Mass Quadrupole Time-of-Flight (Q-TOF) LC/MS, 7800 Quadrupole ICP-MS from Agilent Technologies; 9030 Q-TOF, 8060 Triple Quadrupole LC/MS, 8050 Triple Quadrupole LC/MS from Shimadzu Corporation; AxION 2 TOF MS, NexION ICP-MS series from PerkinElmer; Pegasus BT GC-TOFMS, Pegasus GC-HRT+4D, Citius LC-HRT from LECO Corporation; 6400 Series Triple Quadrupole LC/MS, 6500 Series Accurate-Mass Quadrupole Time-of-Flight (Q-TOF) LC/MS, 7800 Quadrupole ICP-MS from Agilent Technologies are some examples of ESI mass spectrometers that may be used in embodiments of the invention.

The results from the mass spectrometer include three major elements: time, m/z, and intensity. Time represents the collection of individual mass spectra continuously over a selected time period, determined either by a fixed time (e.g., 1 ms, 2 ms, 10 ms, 0.1 ms) or by a total number of ions collected (e.g., 1 million ions, 2 million ions, 5 million ions). The m/z value denotes the mass to charge ratio of the analytes in the mass spectrometer, measured by the mass-to-charge ratio. The typical mass-to-charge ratio extends up to 10,000 m/z, although some instruments can measure higher m/z ratios, reaching up to a few hundred thousand, such as 200,000 m/z. The charge of the analytes depends on the number of chemical groups capable of accepting a proton or electron. The intensity of the peaks present in a mass spectrum correlates with the amount of analytes present in the sample.

Accordingly, FIG. 20 also shows example mass spectra displaying all of the elements. The top spectrum 2006 is an example of m/z versus intensity. In this example, there are 4 clusters of peaks, each representing a specific analyte. Several peaks shown are due to isotopic distribution, which refers to the pattern of peaks corresponding to different isotopes of an element within a molecule. Isotopes are variants of an element that have the same number of protons but different numbers of neutrons, leading to slight differences in mass. This results in multiple peaks in the mass spectrum, each representing a different isotopic composition of the molecule. For example, the element carbon has two stable isotopes, carbon-12 (¹²C) and carbon-13 (¹³C), with natural abundances of approximately 98.9% and 1.1%, respectively. Therefore, a molecule containing carbon may exhibit a characteristic isotopic pattern in its mass spectrum, with peaks corresponding to the different isotopes of carbon present. Similarly, other elements such as hydrogen, nitrogen, and oxygen also display isotopic distributions in mass spectra, reflecting the natural abundance of their isotopes.

In proteomics, identification and characterization of peptide and protein is generally performed using monoisotopic mass. Mono-isotopic mass refers to the mass calculated based on the exact atomic masses of the most abundant isotopes of each element present in the molecule. In mass spectrometry, the monoisotopic mass is often used as a reference mass for matching experimental mass spectra with theoretical peptide or protein sequences. Accurate determination of the monoisotopic mass of peptides detected in mass spectrometry experiments provides various benefits, including for instance, (1) enabling identification of proteins, (2) elucidating post-translational modifications, and/or (3) studying protein-protein interactions. Such analysis and determinations can therefore advance the understanding of biological systems at the molecular level.

Graph 2007 shows the extracted chromatogram of the monoisotopic peak of each analyte. An extracted ion chromatogram (EIC) is a graphical representation used in chromatography, particularly in mass spectrometry-based analyses, to isolate and visualize the chromatographic elution profile of a specific ion of interest. EIC allows to focus on a particular analyte by extracting the chromatographic signal corresponding to its specific mass-to-charge ratio (m/z). The resulting chromatogram provides a clear depiction of the elution behavior of the target ion, facilitating its identification and quantification in the sample. The area under the curve of an extracted ion chromatogram is proportional to the amount of analyte present in the sample.

In addition to the time, m/z, and intensity element, most of the mass spectrometers can do tandem mass spectrometry or MS/MS analysis. Such analysis generally involves the sequential use of two or more mass analyzers to provide detailed structural and quantitative information about molecules in a sample. In a typical setup, the first mass analyzer selects and isolates a precursor ion of interest from the sample mixture based on its mass-to-charge ratio (m/z). The isolated precursor ion is then subjected to fragmentation, either through collision-induced dissociation (CID), electron capture dissociation (ECD), or other fragmentation techniques, generating a series of fragment ions. These fragment ions are then analyzed by the second mass analyzer, allowing for the determination of the molecular structure, identification of functional groups, and elucidation of the sequence of biomolecules.

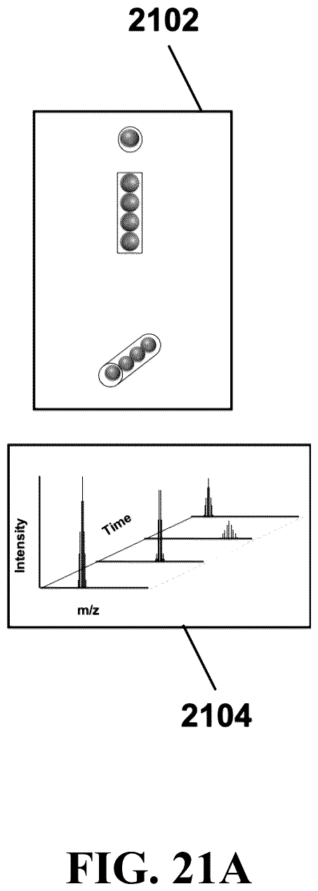

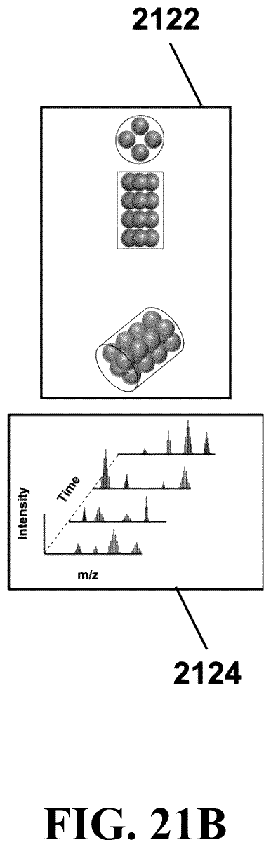

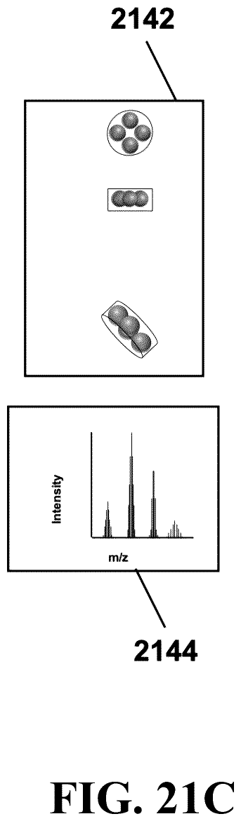

FIGS. 21A-21C depict the example mass spectra obtained with different bead arrangements inside the chambers. In FIG. 21A, the beads are packed one per layer, as shown in the various views 2102. The resulting mass spectrum 2104 demonstrates that the analytes are separated along the time axis. FIG. 21B depicts multiple beads per layer arranged in several layers, as shown in the various views 2122. The resulting mass spectrum 2124 shows the separation of analytes along both the m/z axis and time axis. In the case of a single layer as depicted in FIG. 21C depicts a single layer of beads, as shown in the various views 2142. The analytes are eluted simultaneously but separated along the m/z axis, as shown in the resulting mass spectrum 2144.

A skilled artisan will appreciate that additional features of embodiments of the invention are possible, including, for instance, (1) integrating sample processing upstream of bead handling, (2) encompassing one or more steps (e.g., pre-treatment steps) described in Table 1, (3) incubation of beads with the sample, combined with subsequent bead-washing, (4) modification of one or more eluted analytes (e.g., eluted proteins) after elution, (5) adding one or more specific tags or barcodes to one or more eluted analytes, and/or (6) partial or complete fragmentation and/or digestion of one or more eluted analytes, which could then be utilized in further analysis (e.g., mass spectrometry-based protein sequencing).

These and other objectives and features of the invention are apparent in the disclosure, which includes the above and ongoing written specifications.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

What is claimed is:

1. An analytical apparatus for rapid, continuous, and scalable elution of analytes from a plurality of beads, comprising:
one or more bead handlers;
a bead feeder;
a plurality of chambers, each chamber in the plurality of chambers comprising (i) a first opening fluidically connected to an interior of the chamber, and (ii) a second opening fluidly connected to the interior of the chamber;
a plurality of fittings that are fluidly connected to ends of the plurality of chambers;
one or more pumps attached to the ends of the plurality of chambers;
one or more waste collection units; and
one or more connectors that are connected to one or more analyte analysis devices.

2. The apparatus of claim 1, wherein the one or more bead handlers further comprises:
a bead storage area for storing the plurality of beads; and
a bead manipulator for moving the plurality of beads from the bead storage area to the bead feeder,
wherein the bead storage area further comprises a sample storage area for storing one or more samples.

3. The apparatus of claim 2, wherein the sample storage area is physically sized to hold (i) one or more multi-well plates in which the one or more samples are disposed, and/or (ii) one or more vials in which the one or more samples are disposed.

4. The apparatus of claim 2, wherein the bead manipulator comprises a pipetting unit, and wherein the pipetting unit comprises (i) a tip, and (ii) an aspirator that provides suction through the tip.

5. The apparatus of claim 2, wherein the bead manipulator comprises one or more magnets for picking up at least one bead in the plurality of beads.

6. The apparatus of claim 1, wherein the bead feeder is fluidly connected to the plurality of chambers via inlet ports, and wherein the bead feeder comprises one or more valves for controlling flow of the plurality of beads into the plurality of chambers.

7. The apparatus of claim 1, wherein the ends of the plurality of chambers comprises inlet ends and outlet ends, wherein the plurality of fittings comprises (i) inlet fittings fluidly connected to the inlet ends, and (ii) outlet fittings fluidly connected to the outlet ends.

8. The apparatus of claim 1, wherein one or more pumps are attached to (i) inlet ports of the plurality of chambers, and/or (ii) outlet ports of the plurality of chambers.

9. The apparatus of claim 1, wherein the plurality of chambers are disposed on a rotating device, wherein the interior comprises a hollow portion for receiving the plurality of beads from the bead feeder, and wherein a size of each chamber in the plurality of chambers ranges from about 0.1 μl to about 5 ml.

10. The apparatus of claim 1, wherein the one or more pumps are operatively connected to a control unit configured to regulate (i) flow rate of elution from the plurality of beads, and (ii) timing of elution from the plurality of beads, and wherein the one or more pumps are configured to be reversible to enable bidirectional flow through the plurality of chambers.

11. A method for rapid, continuous, and scalable elution of analytes from a plurality of beads, the method comprising:
for a plurality of bead sets, each of the plurality of bead sets comprising a plurality of beads and one or more associated analytes:
extracting, by one or more bead handlers, the plurality of beads from a bead storage area;
moving, by the one or more bead handlers, the plurality of beads from the bead storage area to a bead feeder, wherein the bead feeder is fluidically connected to at least one chamber in a plurality of chambers;
depositing, by the one or more bead handlers, the plurality of beads into the bead feeder;
moving, by the bead feeder, the plurality of beads into the plurality of chambers;
eluting, by one or more elution solutions, the one or more associated analytes from the plurality of beads;
removing, after the eluting, the plurality of beads from the plurality of chambers; and
transferring one or more associated analytes to one or more analyte analysis devices.

12. The method of claim 11, wherein the removing further comprises moving the plurality of beads from the plurality of chambers to one or more waste collection units.

13. The method of claim 11, further comprising analyzing, by the one or more analyte analysis devices, the one or more associated analytes,
wherein one or more analyte analysis devices comprises a mass spectrometer.

14. The method of claim 13, wherein the analyzing further comprises quantifying and/or identifying one or more associated analytes.

15. A method for rapid, continuous, and scalable elution of analytes from a plurality of beads, the method comprising:
a plurality of repeatable cycles, each cycle in the plurality of cycles comprising:
picking a bead set from a bead storage area;
repositioning the bead set from the bead storage area to one or more chambers;
eluting one or more analytes from the bead set in the one or more chambers to an elution solvent, to generate eluted beads;
removing the eluted beads from the one or more chambers to a waste collection unit; and
after the removing, cleaning one or more chambers.

16. The method of claim 15, wherein each cycle in the plurality of repeatable cycles is repeated for a plurality of bead sets.

17. The method of claim 15, wherein the picking, the eluting, the removing, and the cleaning are performed simultaneously for different bead sets.

18. The method of claim 15, wherein the eluting of the one or more analytes is performed at least partially by pumping the elution solvent through one or more pumps, and wherein the one or more pumps are adjustable to control the rate of elution.

19. The method of claim 15, wherein each bead within the bead set is associated with one or more compounds selected from the group consisting of: one or more nucleic acids, one or more proteins, one or more protein complexes, one or more modified proteins, one or more peptides, one or more carbohydrates, one or more metabolites, one or more lipids, one or more enzymes, one or more protein fragments, one or more nucleic acid fragments, one or more carbohydrate fragments, one or more lipid fragments, one or more pharmacological substances, one or more drugs, one or more pro-drugs, one or more small molecules, one or more chemical compounds, one or more biological compounds, one or more atoms, one or more molecules, and combinations thereof.

20. The method of claim 15, wherein each bead in the bead set comprises at least one chemically distinct molecule configured to identify each bead.
